(19) United States
(12) Patent Application Publication
(10) Pub. No.: US 2008/0269209 A1
(43) Pub. Date: Oct. 30, 2008

(54) Pyrazoloisoquinoline Derivatives

(75) Inventors: Carmen Almansa Rosales, Barcelona (ES); Marina Virgili Bernado, Barcelona (ES)

Correspondence Address: ROTHWELL, FIGG, ERNST & MANBECK, P.C., 1425 K STREET, N.W., SUITE 800, WASHINGTON, DC, 20005, US

(73) Assignee: PALAU PHARMA, S.A., Palau-solità i Plegamans (ES)

(21) Appl. No.: 12/094,718

(22) PCT Filed: Nov. 23, 2006
(86) PCT No.: PCT/EP2006/068815

§ 371 Date: May 22, 2008

(30) Foreign Application Priority Data
Nov. 25, 2005 (EP) 05381056.0

(51) Int. Cl. A61K 31/5377; A61K 31/437; A61P 29/00; C07D 471/04

(52) U.S. Cl. 514/232.8; 546/82; 514/293; 544/126

(57) ABSTRACT

New compounds of formula I, wherein the meanings for the various substituents are as disclosed in the description. These compounds are useful as p38 kinase inhibitors. (I)

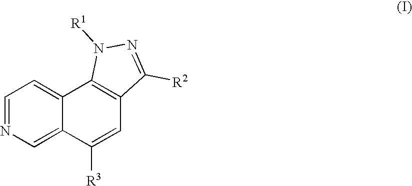

### FIELD OF THE INVENTION

The present invention relates to a new series of pyrazoloisoquinoline derivatives, a process to prepare them, pharmaceutical compositions containing these compounds and their use in therapy.

### BACKGROUND OF THE INVENTION

Kinases are proteins involved in different cellular responses to external signals. In the Nineties, a new family of kinases called MAPK (mitogen-activated protein kinases) was discovered. MAPK activate their substrates by phosphorylation in serine and threonine residues.

MAPK are activated by other kinases in response to a wide range of signals including growth factors, pro-inflammatory cytokines, UV radiation, endotoxins and osmotic stress. Once they are activated, MAPK activate by phosphorylation other kinases or proteins, such as transcription factors, which, ultimately, induce an increase or a decrease in expression of a specific gene or group of genes.

The MAPK family includes kinases such as p38, ERK (extracellular-regulated protein kinase) and JNK (C-Jun N-terminal kinase).

p38 knase plays a crucial role in cellular response to stress and in the activation pathway in the synthesis of numerous cytokines, especially tumor necrosis factor (TNF-α), interleukin-1 (IL-1), interleukin-6 (IL-6) and interleukin-8 (IL-8).

IL-1 and TNF-α are produced by macrophages and monocytes and are involved in the mediation of immunoregulation processes and other physiopathological conditions. For example, elevated levels of TNF-α are associated with inflammatory and autoimmune diseases and with processes that trigger the degradation of connective and bone tissue such as rheumatoid arthritis, osteoarthritis, diabetes, inflammatory bowel disease and sepsis.

Thus, it is believed that p38 kinase inhibitors can be useful to treat or prevent diseases mediated by cytokines such as IL-1 and TNF-α, such as the ones mentioned above.

On the other hand, it has also been found that p38 inhibitors inhibit other pro-inflammatory proteins such as IL-6, IL-8, interferon-y and GM-CSF (granulocyte-macrophage colony-stimulating factor). Moreover, in recent studies it has been found that p38 inhibitors not only block cytokine synthesis but also the cascade of signals that these induce, such as induction of the cyclooxygenase-2 enzyme (COX-2).

Accordingly, it would be desirable to provide novel compounds which are capable of inhibiting p38.

### DESCRIPTION OF THE INVENTION

One aspect of the present invention relates to the compounds of general formula I

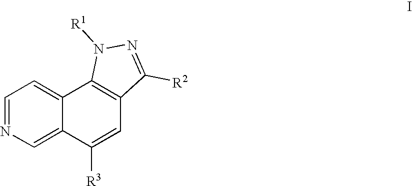

wherein:
R¹ represents phenyl optionally substituted with one or more substituents selected from R^{a}, halogen, —CN, —OH and —OR^{a};
R² represents H, halogen, —OR^{b′}, —NO₂, —CN, —COR^{b′}, —CO₂R^{b′}, —CONR^{b′}R^{c′}, —NR^{b′}R^{d}, —NR^{c′}COR^{b′}, —NR^{c′}CONR^{b′}R^{c′}, —NR^{c′}CO₂R^{b}, —NR^{c′}SO₂R^{b}, Cy¹, —(C₁₋₄alkyl)-Cy¹ or C₁₋₄alkyl optionally substituted with one or more substituents selected from halogen, —OR^{e′}, —NO₂, —CN, —COR^{e′}, —CO₂R^{e′}, —CONR^{c′}R^{e′}, —NR^{d}R^{e′}, —NR^{c′}COR^{e′}, —NR^{c′}CONR^{c′}R^{e′}, —NR^{c′}CO₂R^{e} and —NR^{c′}SO₂R^{e};
R³ represents halogen, —OR^{f′}, —NO₂, —CN, —COR^{f′}, —CO₂R^{f′}, —CONR^{c′}R^{f′}, —NR^{d}R^{f′}, —NR^{c′}COR^{f′}, —NR^{c′}CONR^{c′}R^{f′}, —NR^{c′}CO₂R^{f}, —NR^{c′}SO₂R^{f}, Cy², —(C₁₋₄alkyl)-Cy¹ or —(C₁₋₄alkyl)-NR^{c′}R^{f′};
Cy¹ represents Cy optionally substituted with one or more substituents selected from R^{c} and R^{g};
Cy² represents Cy optionally substituted with one or more substituents selected from R^{b} and R^{h};
each R^{a} independently represents C₁₋₄alkyl or haloC₁₋₄alkyl;
each R^{b} independently represents Cy¹, —(C₁₋₄alkyl)-Cy¹ or C₁₋₄alkyl optionally substituted with one or more substituents R^{g};
each R^{b′} independently represents H or R^{b};
each R^{c} independently represents C₁₋₄alkyl, haloC₁₋₄alkyl or hydroxyC₁₋₄alkyl;
each R^{c′} independently represents H or R^{c};
each R^{d} independently represents R^{c′} or —COR^{c};
each R^{e} independently represents R^{c} or Cy¹;
each R^{e′} independently represents H or R^{e};
each R^{f} independently represents R^{e} or —(C₁₋₄alkyl)-Cy¹;
each R^{f′} independently represents H or R^{f};
each R^{g} independently represents halogen, —OR^{c′}, —NO₂, —CN, —COR^{c′}, —CO₂R^{c′}, —CONR^{c′}R^{c′}, —NR^{c′}R^{c′}, —NR^{c′}COR^{c′}, —NR^{c′}CONR^{c′}R^{c′}, —NR^{c′}CO₂R^{c}, —NR^{c′}SO₂R^{c}, —SR^{c′}, —SOR^{c}, —SO₂R^{c} or —SO₂NR^{c′}R^{c′};
each R^{h} independently represents halogen, —OR^{b′}, —NO₂, —CN, —COR^{b′}, —CO₂R^{b′}, —CONR^{b′}R^{c′}, —NR^{b′}R^{d}, —NR^{c′}COR^{b′}, —NR^{c′}CONR^{b′}R^{c′}, —NR^{c′}CO₂R^{b}, —NR^{c′}SO₂R^{b}; —SR^{b′}, —SOR^{b}, —SO₂R^{b} or —SO₂NR^{b′}R^{c′}; and
Cy in the above definitions represents a saturated, partially unsaturated or aromatic 3- to 7-membered monocyclic or 8- to 12-membered bicyclic carbocyclic ring, which optionally contains from 1 to 4 heteroatoms selected from N, S and O, wherein one or more C, N or S can be optionally oxidized forming CO, N⁺O⁻, SO or SO₂ respectively, and wherein said ring or rings can be linked to the rest of the molecule through a carbon or a nitrogen atom.

The present invention also relates to the salts and solvates of the compounds of formula I.

Some compounds of formula I can have chiral centres that can give rise to various stereoisomers. The present invention relates to each of these stereoisomers and also mixtures thereof.

The compounds of formula I are p38 kinase inhibitors and also inhibit the production of cytokines such as TNF-α.

Thus, another aspect of the invention relates to a compound of general formula I

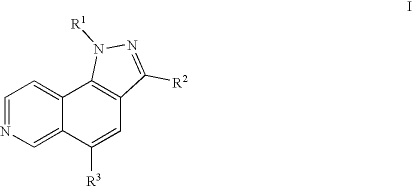

wherein:
R¹ represents phenyl optionally substituted with one or more substituents selected from R^{a}, halogen, —CN, —OH and —OR^{a};
R² represents H, halogen, —OR^{b′}, —NO₂, —CN, —COR^{b′}, —CO₂R^{b′}, —CONR^{b′}R^{c′}, —NR^{b′}R^{d}, —NR^{c′}COR^{b′}, —NR^{c′}CONR^{b′}R^{c′}, —NR^{c′}CO₂R^{b}, —NR^{c′}SO₂R^{b}, Cy¹, —(C₁₋₄alkyl)-Cy¹ or C₁₋₄alkyl optionally substituted with one or more substituents selected from halogen, —OR^{e′}, —NO₂, —CN, —COR^{e′}, —CO₂R^{e′}, —CONR^{c′}R^{e′}, —NR^{d}R^{e′}, —NR^{c′}COR^{e′}, —NR^{c′}CONR^{c′}R^{e′}, —NR^{c′}CO₂R^{e} and —NR^{c′}SO₂R^{e};
R³ represents halogen, —OR^{f′}, —NO₂, —CN, —COR^{f′}, —CO₂R^{f′}, —CONR^{c′}R^{f′}, —NR^{d}R^{f′}, —NR^{c′}COR^{f′}, —NR^{c′}CONR^{c′}R^{f′}, —NR^{c′}CO₂R^{f}, —NR^{c′}SO₂R^{f}, Cy², —(C₁₋₄alkyl)-Cy¹ or —(C₁₋₄alkyl)-NR^{c′}R^{f′};
Cy¹ represents Cy optionally substituted with one or more substituents selected from R^{c} and R^{g};
Cy² represents Cy optionally substituted with one or more substituents selected from R^{b} and R^{h};
each R^{a} independently represents C₁₋₄alkyl or haloC₁₋₄alkyl;
each R^{b} independently represents Cy¹, —(C₁₋₄alkyl)-Cy¹ or C₁₋₄alkyl optionally substituted with one or more substituents R^{g};
each R^{b′} independently represents H or R^{b};
each R^{c} independently represents C₁₋₄alkyl, haloC₁₋₄alkyl or hydroxyC₁₋₄alkyl;
each R^{c′} independently represents H or R^{c};
each R^{d} independently represents R^{c′} or —COR^{c};
each R^{e} independently represents R^{c} or Cy¹;
each R^{e′} independently represents H or R^{e};
each R^{f} independently represents R^{e} or —(C₁₋₄alkyl)-Cy¹;
each R^{f′} independently represents H or R^{f};
each R^{g} independently represents halogen, —OR^{c′}, —NO₂, —CN, —COR^{c′}, —CO₂R^{c′}, —CONR^{c′}R^{c′}, —NR^{c′}R^{c′}, —NR^{c′}COR^{c′}, —NR^{c′}CONR^{c′}R^{c}, —NR^{c′}CO₂R^{c}, —NR^{c′}SO₂R^{c}, —SR^{c′}, —SOR^{c}, —SO₂R^{c} or —SO₂NR^{c′}R^{c′};
each R^{h} independently represents halogen, —OR^{b′}, —NO₂, —CN, —COR^{b′}, —CO₂R^{b′}, —CONR^{b′}R^{c′}, —NR^{b′}R^{d}, —NR^{c′}COR^{b′}, —NR^{c′}CONR^{b′}R^{c′}, —NR^{c′}CO₂R^{b}, —NR^{c′}SO₂R^{b}; —SR^{b′}, —SOR^{b}, —SO₂R^{b} or —SO₂NR^{b′}R^{c′}; and

Cy in the above definitions represents a saturated, partially unsaturated or aromatic 3- to 7-membered monocyclic or 8- to 12-membered bicyclic carbocyclic ring, which optionally contains from 1 to 4 heteroatoms selected from N, S and O, wherein one or more C, N or S can be optionally oxidized forming CO, N⁺O⁻, SO or SO₂ respectively, and wherein said ring or rings can be linked to the rest of the molecule through a carbon or a nitrogen atom;

for use in therapy.

Another aspect of the present invention relates to the pharmaceutical compositions which comprise a compound of formula I or a pharmaceutically acceptable salt thereof and one or more pharmaceutically acceptable excipients.

Another aspect of the present invention relates to the use of a compound of formula I or a pharmaceutically acceptable salt thereof for the manufacture of a medicament for the treatment or prevention of diseases mediated by p38.

Another aspect of the present invention relates to the use of a compound of formula I or a pharmaceutically acceptable salt thereof for the manufacture of a medicament for the treatment or prevention of diseases mediated by cytokines.

Another aspect of the present invention relates to the use of a compound of formula I or a pharmaceutically acceptable salt thereof for the manufacture of a medicament for the treatment or prevention of diseases mediated by TNF-α, IL-1, IL-6 and/or IL-8.

Another aspect of the present invention relates to the use of a compound of formula I or a pharmaceutically acceptable salt thereof for the manufacture of a medicament for the treatment or prevention of a disease selected from immune, autoimmune and inflammatory diseases, cardiovascular diseases, infectious diseases, bone resorption disorders, neurodegenerative diseases, proliferative diseases and processes associated with the induction of cyclooxygenase-2.

Another aspect of the present invention relates to the use of a compound of formula I or a pharmaceutically acceptable salt thereof for the treatment or prevention of diseases mediated by p38.

Another aspect of the present invention relates to the use of a compound of formula I or a pharmaceutically acceptable salt thereof for the treatment or prevention of diseases mediated by cytokines.

Another aspect of the present invention relates to the use of a compound of formula I or a pharmaceutically acceptable salt thereof for the treatment or prevention of diseases mediated by TNF-α, IL-1, IL-6 and/or IL-8.

Another aspect of the present invention relates to the use of a compound of formula I or a pharmaceutically acceptable salt thereof for the treatment or prevention of a disease selected from immune, autoimmune and inflammatory diseases, cardiovascular diseases, infectious diseases, bone resorption disorders, neurodegenerative diseases, proliferative diseases and processes associated with the induction of cyclooxygenase-2.

Another aspect of the present invention relates to a method of treating or preventing a disease mediated by p38 in a subject in need thereof, especially a human being, which comprises administering to said subject a therapeutically effective amount of a compound of formula I or a pharmaceutically acceptable salt thereof.

Another aspect of the present invention relates to a method of treating or preventing a disease mediated by cytokines in a subject in need thereof, especially a human being, which comprises administering to said subject a therapeutically effective amount of a compound of formula I or a pharmaceutically acceptable salt thereof.

Another aspect of the present invention relates to a method of treating or preventing a disease mediated by TNF-α, IL-1, IL-6 and/or IL-8 in a subject in need thereof, especially a human being, which comprises administering to said subject a therapeutically effective amount of a compound of formula I or a pharmaceutically acceptable salt thereof.

Another aspect of the present invention relates to a method of treating or preventing a disease selected from immune, autoimmune and inflammatory diseases, cardiovascular diseases, infectious diseases, bone resorption disorders, neurodegenerative diseases, proliferative diseases and processes associated with the induction of cyclooxygenase-2 in a subject in need thereof, especially a human being, which comprises administering to said subject a therapeutically effective amount of a compound of formula I or a pharmaceutically acceptable salt thereof.

Another aspect of the present invention relates to a process for the preparation of a compound of formula I as defined above, which comprises: (a) when in a compound of formula I R³ represents halogen, reacting a compound of formula IV

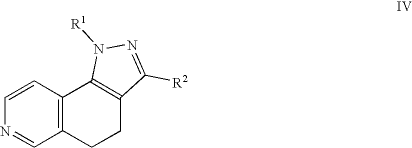

wherein R¹ and R² have the meaning described above, with a suitable halogenating agent; or
(b) when in a compound of formula I R³ represents aryl or heteroaryl optionally substituted with one or more substituents selected from R^{b} and R^{h}, reacting a compound of formula I wherein R³ represents halogen (Ia)

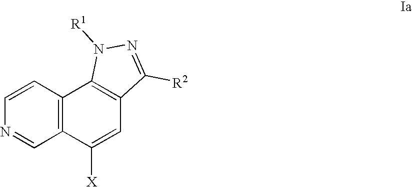

wherein R¹ and R² have the meaning described above and X represents halogen, preferably chloro or bromo, with a boron derivative of formula Cy²-B(ORⁱ)₂ (II) or with a derivative of formula IIa,

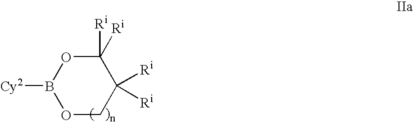

wherein n represents 0 or 1, Cy² represents aryl or heteroaryl optionally substituted with one or more substituents selected from R^{b} and R^{h}, and wherein each Rⁱ independently represents H or C₁₋₄alkyl; or
(c) when in a compound of formula I R³ represents —NR^{f}R^{c′}, reacting a compound of formula la with an amine of formula HNR^{f}R^{c′} (III); or
(d) when in a compound of formula I R³ represents Cy² linked to the central ring through a N atom, reacting a compound of formula la with the corresponding cyclic amine; or
(e) converting, in one or a plurality of steps, a compound of formula I into another compound of formula I.

In the above definitions, the term C₁₋₄alkyl, as a group or part of a group, means a straight or branched alkyl chain which contains from 1 to 4 carbon atoms. Examples include the groups methyl, ethyl, propyl, isopropyl, butyl, isobutyl, sec-butyl and tert-butyl.

A haloC₁₋₄alkyl group means a group resulting from the replacement of one or more hydrogen atoms from a C₁₋₄alkyl group with one or more halogen atoms (i.e. fluoro, chloro, bromo or iodo), which can be the same or different. Examples include, among others, trifluoromethyl, fluoromethyl, 1-chloroethyl, 2-chloroethyl, 1-fluoroethyl, 2-fluoroethyl, 2-bromoethyl, 2-iodoethyl, 2,2,2-trifluoroethyl, pentafluoroethyl, 3-fluoropropyl, 3-chloropropyl, 2,2,3,3-tetrafluoropropyl, 2,2,3,3,3-pentafluoropropyl, heptafluoropropyl, 4-fluorobutyl and nonafluorobutyl.

A hydroxyC₁₋₄alkyl group means a group resulting from the replacement of one or more hydrogen atoms from a C₁₋₄alkyl group with one or more —OH. Examples include, among others, hydroxymethyl, 1-hydroxyethyl, 2-hydroxyethyl, 1,2-dihydroxyethyl, 3-hydroxypropyl and 4-hydroxybutyl.

A —(C₁₋₄alkyl)-Cy¹ group means a group resulting from the replacement of one hydrogen atom from a C₁₋₄alkyl group with one Cy¹ group. Examples include, among others, benzyl, piperidin-1-ylmethyl, piperazin-1-ylmethyl, 1-phenylethyl, 2-phenylethyl, 2-morpholin-4-ylethyl, 3-piperidin-4-ylpropyl and 4-piridin-4-ylbutyl.

A —(C₁₋₄alkyl)-NR^{c′}R^{f′} group means a group resulting from the replacement of one hydrogen atom from a C₁₋₄alkyl group with one —NR^{c′}R^{f′} group. Examples include, among others, aminomethyl, (1-phenylethyl)aminomethyl, benzylaminomethyl, (dimethylamino)methyl, (phenylamino)methyl, 2-aminoethyl, 3-aminopropyl and 4-aminobutyl.

A halogen radical means fluoro, chloro, bromo or iodo.

The term Cy in the definitions of Cy¹ and Cy² means a 3- to 7-membered monocyclic carbocyclic ring or an 8- to 12-membered bicyclic carbocyclic ring which can be partially unsaturated, saturated or aromatic and which can optionally contain from 1 to 4 heteratoms selected from N, S and O. The Cy group can be linked to the rest of the molecule through any available carbon or nitrogen atom. When the Cy group is saturated or partially unsaturated, one or more C or S atoms can be optionally oxidized forming CO, SO or SO₂. When the Cy group is aromatic, one or more N atoms can be optionally oxidized forming N⁺O⁻. The Cy group can be optionally substituted as disclosed above in the definitions of Cy¹ and Cy²; if substituted, the substituents can be the same or different and can be placed on any available position. Examples of Cy groups include, among others, cyclopropyl, cyclobutyl, cyclopentyl, cyclohexyl, cycloheptyl, aziridinyl, oxiranyl, oxetanyl, imidazolidinyl, isothiazolidinyl, isoxazolidinyl, oxazolidinyl, pyrazolidinyl, pyrrolidinyl, thiazolidinyl, dioxanyl, morpholinyl, piperazinyl, piperidinyl, pyranyl, tetrahydropyranyl, azepinyl, oxazinyl, oxazolinyl, pyrrolinyl, thiazolinyl, pyrazolinyl, imidazolinyl, isoxazolinyl, isothiazolinyl, phenyl, naphthyl, 1,2,4-oxadiazolyl, 1,2,4-thiadiazolyl, 1,3,4-oxadiazolyl, 1,3,4-thiadiazolyl, furyl, imidazolyl, isoxazolyl, isothiazolyl, oxazolyl, pyrazolyl, pyrrolyl, thiazolyl, thienyl, 1,2,3-triazolyl, 1,2,4-triazolyl, pyrazinyl, pyridazinyl, pyridyl, pyrimidinyl, benzimidazolyl, benzofuranyl, isobenzofuranyl, benzothiazolyl, benzothiophenyl, isobenzothiophenyl, imidazopyrazinyl, imidazopyridazinyl, imidazopyridinyl, imidazopyrimidinyl, indazolyl, indolyl, isoindolyl, isoquinolinyl, tetrahydroisoquinolinyl, naphthyridinyl, pyrazolopyrazinyl, pyrazolopyridinyl, pyrazolopyrimidinyl, purinyl, quinazolinyl, quinolinyl, quinoxalinyl, 2-oxo-cyclobutyl, 2-oxocyclopentyl, 2-oxocyclohexyl, 2-oxocycloheptyl, 2-oxo-pyrrolidinyl, 2-oxo-piperidinyl, 4-oxo-piperidinyl, 2-oxo-piperazinyl, 2(1H)-pyridonyl, 2(1H)-pyrazinonyl, 2(1H)-pyrimidinonyl, 2(1H)-pyridazinonyl and phthalimidyl.

An aryl group means phenyl or naphthyl and can be optionally substituted as disclosed whenever this term is used, said substituents being placed on any available position of the aryl group.

The term heteroaryl means an aromatic 5- or 6-membered monocyclic or 8- to 12-membered bicyclic ring which contains from 1 to 4 heteroatoms selected from N, S and O, wherein one or more N atoms can be optionally oxidized forming N⁺O⁻. The heteroaryl group can be optionally substituted as disclosed whenever this term is used, said substituents being placed on any available position. The heteroaryl group can be linked to the rest of the molecule through any available carbon or nitrogen atom. Examples of heteroaryl groups include among others 1,2,4-oxadiazolyl, 1,2,4-thiadiazolyl, 1,3,4-oxadiazolyl, 1,3,4-thiadiazolyl, furyl, imidazolyl, isoxazolyl, isothiazolyl, oxazolyl, pyrazolyl, pyrrolyl, thiazolyl, thienyl, 1,2,3-triazolyl, 1,2,4-triazolyl, pyrazinyl, pyridazinyl, pyridyl, pyrimidinyl, benzimidazolyl, benzofuranyl, benzothiazolyl, benzothiophenyl, imidazopyrazinyl, imidazopyridazinyl, imidazopyridinyl, imidazopyrimidinyl, indazolyl, indolyl, isoindolyl, isoquinolinyl, naphthyridinyl, pyrazolopyrazinyl, pyrazolopyridinyl, pyrazolopyrimidinyl, purinyl, quinazolinyl, quinolinyl and quinoxalinyl.

In the above definitions of heteroaryl and Cy, when the specified examples refer to a bicycle in general terms, all possible dispositions of the atoms are included. For example, the term pyrazolopyridinyl is to be understood as including groups such as 1H-pyrazolo[3,4-b]pyridinyl, pyrazolo[1,5-a]pyridinyl, 1H-pyrazolo[3,4-c]pyridinyl, 1H-pyrazolo[4,3-c]pyridinyl and 1H-pyrazolo[4,3-b]pyridinyl; the term imidazopyrazinyl is to be understood as including groups such as 1H-imidazo[4,5-b]pyrazinyl, imidazo[1,2-a]pyrazinyl and imidazo[1,5-a]pyrazinyl and the term pyrazolopyrimidinyl is to be understood as including groups such as 1H-pyrazolo[3,4-d]pyrimidinyl, 1H-pyrazolo[4,3-d]pyrimidinyl, pyrazolo[1,5-a]pyrimidinyl and pyrazolo[1,5-c]pyrimidinyl.

The expression “optionally substituted with one or more” means that a group can be substituted with one or more, preferably with 1, 2, 3 or 4 substituents, more preferably with 1 or 2 substituents, provided that said group has enough positions available susceptible of being substituted. When present, said substituents can be the same or different and can be placed on any available position.

When in a definition of a substituent two or more groups bearing the same numbering are shown (e.g. —NR^{c′}COR^{c′}, —NR^{c′}R^{c′}, —NR^{c′}CONR^{b′}R^{c′}, etc), this does not mean that they have to be identical. Each of them is independently selected from the list of possible meanings provided for that group, and therefore they can be the same or different.

The invention thus relates to the compounds of formula I as defined here above.

In another embodiment, the invention relates to the compounds of formula I wherein R¹ represents phenyl optionally substituted with one or more, preferably 1 or 2, substituents selected from halogen and haloC₁₋₄alkyl.

In another embodiment, the invention relates to the compounds of formula I wherein R¹ represents phenyl substituted with one or more, preferably 1 or 2, substituents selected from halogen and haloC₁₋₄alkyl.

In another embodiment, the invention relates to the compounds of formula I wherein R¹ represents phenyl substituted with one or more, preferably 1 or 2, halogen atoms, preferably fluorine atoms.

In another embodiment, the invention relates to the compounds of formula I wherein R² represents H, halogen, —CONR^{b′}R^{c′}, —NR^{b′}R^{d}, Cy¹ or C₁₋₄alkyl optionally substituted with one or more substituents selected from —OR^{e′} and —NR^{d}R^{e′}.

In another embodiment, the invention relates to the compounds of formula I wherein R² represents H, —CONR^{b′}R^{c′}, —NR^{b′}R^{d}, Cy¹ or C₁₋₄alkyl substituted with one or more substituents selected from —OR^{e′} and —NR^{d}R^{e′}.

In another embodiment, the invention relates to the compounds of formula I wherein R² represents H, —NR^{b′}R^{d}, Cy¹ or C₁₋₄alkyl substituted with one or more substituents selected from —OR^{e′} and —NR^{d}R^{e′}.

In another embodiment, the invention relates to the compounds of formula I wherein R² represents H.

In another embodiment, the invention relates to the compounds of formula I wherein R³ represents halogen, —CN, —CONR^{c′}R^{f′}, —NR^{d}R^{f′}, Cy² or —(C₁₋₄alkyl)-NR^{c′}R^{f′}.

In another embodiment, the invention relates to the compounds of formula I wherein R³ represents —NR^{d}R^{f′}or Cy².

In another embodiment, the invention relates to the compounds of formula I wherein R³ represents Cy².

In another embodiment, the invention relates to the compounds of formula I wherein Cy² represents Cy optionally substituted with one or more substituents selected from C₁₋₄alkyl, haloC₁₋₄alkyl, hydroxyC₁₋₄alkyl, halogen, —OR^{b′}, —NO₂, —CN, —COR^{b′}, —CO₂R^{b′}, —CONR^{b′}R^{c′}, —NR^{b′}R^{d}, —NR^{c′}COR^{b′}, —NR^{c′}CONR^{b′}R^{c′}, —NR^{c′}CO₂R^{b}, —NR^{c′}SO₂R^{b}; —SR^{b′}, —SOR^{b}, —SO₂R^{b} and —SO₂NR^{b′}R^{c′}.

In another embodiment, the invention relates to the compounds of formula I wherein R³ represents a saturated, partially unsaturated or aromatic 3- to 7-membered monocyclic or 8- to 12-membered bicyclic carbocyclic ring, which optionally contains from 1 to 4 heteroatoms selected from N, S and O, and which can be optionally substituted with one or more substituents selected from R^{b} and R^{h}.

In another embodiment, the invention relates to the compounds of formula I wherein R³ represents a saturated, partially unsaturated or aromatic 6-membered monocyclic carbocyclic ring, which optionally contains from 1 to 4 heteroatoms selected from N, S and O, and which can be optionally substituted with one or more substituents selected from R^{b} and R^{h}.

In another embodiment, the invention relates to the compounds of formula I wherein R³ represents a saturated, partially unsaturated or aromatic 6-membered monocyclic carbocyclic ring, which optionally contains 1 or 2 heteroatoms selected from N, S and O, and wherein one or more C, N or S atoms can be optionally oxidized forming CO, N⁺O⁻, SO or SO₂ respectively, wherein R³ can be optionally substituted with one or more substituents selected from R^{b} and R^{h}.

In another embodiment, the invention relates to the compounds of formula I wherein R³ represents:

(i) an aromatic 6-membered carbocyclic ring optionally containing 1 or 2 N atoms, or

(ii) a saturated 6-membered heterocyclic ring containing 1 or 2 heteroatoms selected from N, S and O and wherein one or more C or S atoms can be optionally oxidized forming CO, SO or SO₂ respectively,

wherein R³ can be optionally substituted with one or more substituents selected from R^{b} and R^{h}.

In another embodiment, the invention relates to the compounds of formula I wherein R³ represents:

(i) an aromatic 6-membered carbocyclic ring optionally containing 1 or 2 N atoms, or

(ii) a saturated 6-membered heterocyclic ring containing 1 or 2 heteroatoms selected from N, S and O and wherein one or more C or S atoms can be optionally oxidized forming CO, SO or SO₂ respectively,

wherein R³ can be optionally substituted with one or more substituents selected from C₁₋₄alkyl, haloC₁₋₄alkyl, hydroxyC₁₋₄alkyl, halogen, —OR^{b′}, —NO₂, —CN, —COR^{b′}, —CO₂R^{b′}, —CONR^{b′}R^{c′}, —NR^{b′}R^{d}, —NR^{c′}COR^{b′}, —NR^{c′}CONR^{b′}R^{c′}, —NR^{c′}CO₂R^{b}, —NR^{c′}SO₂R^{b}; —SR^{b′}, —SOR^{b}, —SO₂R^{b} and —SO₂NR^{b′}R^{c′}.

In another embodiment, the invention relates to the compounds of formula I wherein R³ represents morpholinyl, piperazinyl, 4-oxo-piperidinyl, phenyl or pyridyl, wherein R³ can be optionally substituted with one or more substituents selected from R^{b} and R^{h}.

In another embodiment, the invention relates to the compounds of formula I wherein R³ represents Cy² and R¹ represents phenyl substituted with one or more, preferably 1 or 2, substituents selected from halogen and haloC₁₋₄alkyl.

In another embodiment, the invention relates to the compounds of formula I wherein R³ represents Cy² and R¹ represents phenyl substituted with one or more, preferably 1 or 2, halogen atoms, preferably fluorine atoms.

In another embodiment, the invention relates to the compounds of formula I wherein R³ represents Cy²; R¹ represents phenyl substituted with one or more, preferably 1 or 2, halogen atoms, preferably fluorine atoms; and R² represents H, —CONR^{b′}R^{c′}, —NR^{b′}R^{d}, Cy¹ or C₁₋₄alkyl substituted with one or more substituents selected from —OR^{e′} and —NR^{d}R^{e′}.

In another embodiment, the invention relates to the compounds of formula I wherein:

R³ represents a saturated, partially unsaturated or aromatic 6-membered monocyclic carbocyclic ring, which optionally contains 1 or 2 heteroatoms selected from N, S and O, and wherein one or more C, N or S atoms can be optionally oxidized forming CO, N⁺O⁻, SO or SO₂ respectively, wherein R³ can be optionally substituted with one or more substituents selected from R^{b} and R^{h};

R¹ represents phenyl substituted with one or more, preferably 1 or 2, halogen atoms, preferably fluorine atoms; and

R² represents H, —CONR^{b′}R^{c′}, —NR^{b′}R^{d}, Cy¹ or C₁₋₄alkyl substituted with one or more substituents selected from —OR^{e′} and —NR^{d}R^{e′}.

Furthermore, all possible combinations of the above-mentioned embodiments form also part of this invention.

In a further embodiment, the invention relates to compounds according to formula I above which provide more than 50% inhibition of p38 activity at 10 μM, more preferably at 1 μM and still more preferably at 0.1 μM, in a p38 assay such as the one described in Test 3.

In a further embodiment, the invention relates to a compound according to formula I selected from:
5-Bromo-1-(4-fluorophenyl)pyrazolo[3,4-f]isoquinoline;
1-(4-Fluorophenyl)-5-[4-(tetrahydropyran-2-yloxy)phenyl]pyrazolo[3,4-f]isoquinoline;
1-(4-Fluorophenyl)-5-(4-pyridyl)pyrazolo[3,4-f]isoquinoline;
1-(4-Fluorophenyl)-5-phenyl-pyrazolo[3,4-f]isoquinoline;
5-(2-Chlorophenyl)-1-(4-fluorophenyl)pyrazolo[3,4-f]isoquinoline;
1-(4-Fluorophenyl)-5-(3-pyridyl)-pyrazolo[3,4-f]isoquinoline;
5-(4-Aminophenyl)-1-(4-fluorophenyl)pyrazolo[3,4-f]isoquinoline;
[1-(4-Fluorophenyl)-5-(3-pyridyl)pyrazolo[3,4-f]isoquinolin-3-yl]methanol;
[1-(4-Fluorophenyl)-5-phenylpyrazolo[3,4-f]isoquinolin-3-yl]methanol;
5-(3-Pyridyl)-1-(3-trifluoromethylphenyl)pyrazolo[3,4-f]isoquinoline;
3-Bromo-1-(4-fluorophenyl)-5-(3-pyridyl)pyrazolo[3,4-f]isoquinoline;
3-Aminomethyl-1-(4-fluorophenyl)-5-phenylpyrazolo[3,4-f]isoquinoline;
4-[1-(4-Fluorophenyl)pyrazolo[3,4-f]isoquinolin-5-yl]benzoic acid;
3-Amino-1-(4-fluorophenyl)-5-(3-pyridyl)pyrazolo[3,4-f]isoquinoline;
1-(4-Fluorophenyl)pyrazolo[3,4-f]isoquinolin-5-carbonitrile;
5-Aminomethyl-1-(4-fluorophenyl)pyrazolo[3,4-f]isoquinoline;
(1S)-1-(4-fluorophenyl)-5-[(1-phenylethyl)amino]pyrazolo[3,4-f]isoquinoline;
1-(4-Fluorophenyl)-5-(phenylamino)-pyrazolo[3,4-f]isoquinoline;
1-(4-Fluorophenyl)-5-(morpholin-4-yl)pyrazolo[3,4-f]isoquinoline;
5-(4-Acetylpiperazin-1-yl)-1-(4-fluorophenyl)pyrazolo[3,4-f]isoquinoline;
1-(4-Fluorophenyl)-5-(4-methylpiperazin-1-yl)pyrazolo[3,4-f]isoquinoline;
[1-(4-Fluorophenyl)pyrazolo[3,4-f]isoquinolin-5-yl]piperidin-4-one;
1-(4-Fluorophenyl)pyrazolo[3,4-f]isoquinolin-5-carboxamide;
1-(4-Fluorophenyl)-5-(3-pyridyl)pyrazolo[3,4f]isoquinolin-3-carboxamide;
1-(4-Fluorophenyl)-3-[(4-methylsulfinylbenzyl)amino]-5-(3-pyridyl)pyrazolo[3,4-f]isoquinoline;
1-(4-Fluorophenyl)-3-(4-methylsulfinylphenyl)-5-(3-pyridyl)pyrazolo[3,4-f]isoquinoline;
1-(4-Fluorophenyl)-5-(piperazin-1-yl)pyrazolo[3,4-f]isoquinoline;
1-(4-Fluorophenyl)-3-[(4-piperidylmethyl)amino]-5-(3-pyridyl)pyrazolo[3,4-f]isoquinoline;
1-(4-Fluorophenyl)-5-phenyl-3-[(4-piperidyl)aminomethyl]pyrazolo[3,4-f]isoquinoline;
1-[4-[1-(4-Fluorophenyl)pyrazolo[3,4-f]isoquinolin-5-yl]piperazin-1-yl]-2-hydroxyethanone;
4-[1-(4-Fluorophenyl)pyrazolo[3,4-f]isoquinolin-5-yl]phenol;
[4-[1-(4-Fluorophenyl)pyrazolo[3,4-f]isoquinolin-5-yl]phenyl]methanol;
3-(1,1-Dioxothiomorpholin-4-yl)-1-(4-fluorophenyl)-5-(3-pyridyl)pyrazolo[3,4-f]isoquinoline;
1-(4-Fluorophenyl)-3-(4-piperidylamino)-5-(3-pyridyl)pyrazolo[3,4-f]isoquinoline;
5-[4-Acetylpiperazin-1-yl]-1-(3-trifluoromethylphenyl)pyrazolo[3,4-f]isoquinoline; and
5-[4-Methylpiperazin-1-yl]-1-(3-trifluoromethylphenyl)pyrazolo[3,4-f]isoquinoline.

The compounds of the present invention may contain one or more basic nitrogens and may, therefore, form salts with organic or inorganic acids. Examples of these salts include: salts with inorganic acids such as hydrochloric acid, hydrobromic acid, hydroiodic acid, nitric acid, perchloric acid, sulfuric acid or phosphoric acid; and salts with organic acids such as methanesulfonic acid, trifluoromethanesulfonic acid, ethanesulfonic acid, benzenesulfonic acid, p-toluenesulfonic acid, fumaric acid, oxalic acid, acetic acid, maleic acid, ascorbic acid, citric acid, lactic acid, tartaric acid, malonic acid, glycolic acid, succinic acid and propionic acid, among others. Some of the compounds of the present invention may contain one or more acidic protons and, therefore, they may also form salts with bases. Examples of these salts include: salts with inorganic cations such as sodium, potassium, calcium, magnesium, lithium, aluminium, zinc, etc; and salts formed with pharmaceutically acceptable amines such as ammonia, alkylamines, hydroxylalkylamines, lysine, arginine, N-methylglucamine, procaine and the like.

There is no limitation on the type of salt that can be used, provided that these are pharmaceutically acceptable when they are used for therapeutic purposes. The term pharmaceutically acceptable salt represents those salts which are, according to medical judgement, suitable for use in contact with the tissues of humans and other mammals without undue toxicity, irritation, allergic response and the like. Pharmaceutically acceptable salts are well known in the art.

The salts of a compound of formula I can be obtained during the final isolation and purification of the compounds of the invention or can be prepared by treating a compound of formula I with a sufficient amount of the desired acid or base to give the salt in a conventional manner. The salts of the compounds of formula I can be converted into other salts of the compounds of formula I by ion exchange using ionic exchange resins.

The compounds of formula I and their salts may differ in some physical properties but they are equivalent for the purposes of the present invention. All salts of the compounds of formula I are included within the scope of the invention.

The compounds of the present invention may form complexes with solvents in which they are reacted or from which they are precipitated or crystallized. These complexes are known as solvates. As used herein, the term solvate refers to a complex of variable stoichiometry formed by a solute (a compound of formula I or a salt thereof) and a solvent. Examples of solvents include pharmaceutically acceptable solvents such as water, ethanol and the like. A complex with water is known as a hydrate. Solvates of compounds of the invention (or salts thereof), including hydrates, are included within the scope of the invention.

Some of the compounds of the present invention may exist as several diastereoisomers and/or several optical isomers. Diastereoisomers can be separated by conventional techniques such as chromatography or fractional crystallization. Optical isomers can be resolved by conventional techniques of optical resolution to give optically pure isomers. This resolution can be carried out on any chiral synthetic intermediate or on products of general formula I. Optically pure isomers can also be individually obtained using enantiospecific synthesis. The present invention covers all isomers and mixtures thereof (for example racemic mixtures) whether obtained by synthesis and also by physically mixing them.

The compounds of formula I can be obtained by following the processes described below. As it will be obvious to one skilled in the art, the exact method used to prepare a given compound may vary depending on its chemical structure. Moreover, in some of the processes described below it may be necessary or advisable to protect the reactive or labile groups by conventional protective groups. Both the nature of these protective groups and the procedures for their introduction or removal are well known in the art (see for example Greene T. W. and Wuts P. G. M, “Protective Groups in Organic Synthesis”, John Wiley & Sons, 3^{rd} edition, 1999). As an example, as protective groups of an amino function tert-butoxycarbonyl (Boc) or benzyl (Bn) groups can be used. Carboxyl groups can be protected for example in the form of C₁₋₄ alkyl esters or arylalkyl esters, such as benzyl, while hydroxyl groups can be protected for example with tetrahydropyranyl (THP) or benzyl (Bn) groups. Whenever a protective group is present, a later deprotection step will be required, which can be performed under standard conditions in organic synthesis, such as those described in the above-mentioned reference.

Most of the compounds of formula I can be obtained from a compound of formula I wherein R³ represents halogen (Ia), as shown in the following scheme:

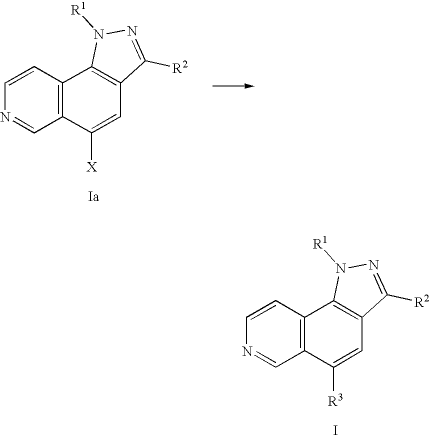

wherein R¹, R² and R³ have the meaning described above in connection with a compound of general formula I and X represents halogen, preferably chloro or bromo.

Thus, for example, compounds of formula I wherein R³ represents aryl or heteroaryl optionally substituted with one or more substituents selected from R^{b} and R^{h} (compounds lb), can be prepared by reacting a compound of formula la with a boron derivative of formula Cy²-B(ORⁱ)₂ (II) or with a derivative of formula IIa,

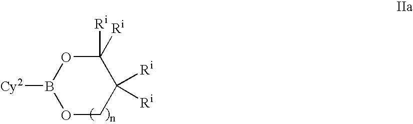

wherein n represents 0 or 1, Cy² represents aryl or heteroaryl optionally substituted with one or more substituents selected from R^{b} and R^{h} and wherein each Rⁱ independently represents H or C₁₋₄alkyl. This reaction is carried out in the presence of a base, such as K₂CO₃, Na₂CO₃ or K₃PO₄, and a palladium catalyst, such as Pd(PPh₃)₄, in a solvent such as dimethoxyethane, dioxane, diglyme or dimethylformamide and heating, preferably at reflux.

Likewise, compounds of formula I wherein R³ represents —NR^{f}R^{c′} (compounds Ic), wherein R^{c′} and R^{f} have the meaning described in general formula I, can be conveniently prepared by reacting a compound of formula la with an amine of formula HNR^{f}R^{c′} (III). This reaction can be carried out in the presence of a base, such as Cs₂CO₃ or sodium tert-butoxide, in the presence of a palladium catalyst, such as palladium acetate (II), and a phosphine such as 2,2′-bis(diphenylphosphino)-1,1′-binaphthyl, in a solvent such as toluene.

Compounds of formula I wherein R³ represents Cy² linked to the central ring through a N atom (compounds Id) can also be conveniently prepared by reacting a compound of formula la with the corresponding cyclic amine (i.e. Cy² wherein the N atom linked to the central ring is present as NH). This reaction is carried out under the same conditions described above for the conversion of a compound la into a compound Ic.

The compounds of formula la can be obtained starting from a compound of formula IV, as shown in the following scheme:

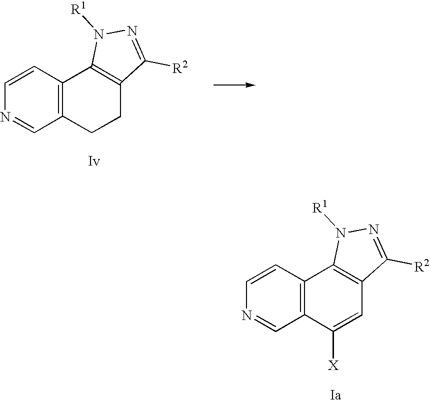

wherein R¹ and R² have the meaning described above and X represents halogen, preferably chloro or bromo. This reaction can be carried out in the presence of a suitable halogenating agent such as Br₂ in a suitable solvent such as trimethyl phosphate, or N-bromosuccinimide optionally in the presence of a radical initiator such as 2,2′-azobis(2-methylbutyronitrile) or benzoyl peroxide in a mixture of CCl₄-CHCl₃, and at a suitable temperature comprised between room temperature and the temperature of the boiling point of the solvent.

Compounds of formula IV can be obtained by reacting a compound of formula V with a compound of formula VI, as shown in the following scheme:

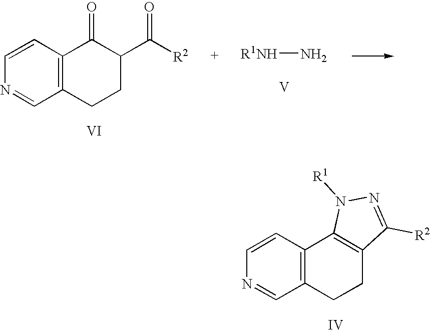

wherein R¹ and R² have the meaning described in general formula I. This reaction can be carried out preferably in the presence of catalytic amounts of an inorganic acid, such as H₂SO₄, in a suitable solvent such as for example 2,2,2-trifluoroethanol or ethanol, and heating, preferably at reflux; or alternatively, this reaction can be carried out in an organic acid, such as acetic acid.

Compounds of formula VI can be prepared by acylating a ketone of formula VIII with a compound of formula VII as shown in the following scheme:

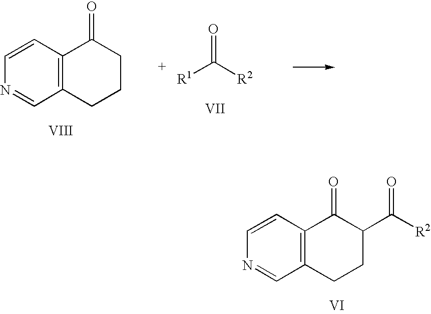

wherein R¹ represents —O—C₁₋₄alkyl, —N(CH₃)OCH₃ or halogen, preferably chloro, and R² has the meaning described in general formula I. This reaction can be carried out in the presence of a base, such as sodium methoxide, lithium bis(trimethylsilyl)amide, sodium bis(trimethylsilyl)amide, lithium diisopropylamide, in a suitable solvent, such as for example dimethoxyethane, tetrahydrofuran or diethyl ether, and at a temperature comprised preferably between −78° C. and room temperature.

The compound of formula VIII has been described in the literature and can be obtained, among others, by the synthetic route shown in J. Epsztajn, J. Chem. Soc. Perkin Trans. 1 1985, 213-220.

Compounds of formula II, IIa, III, V and VII are commercially available or can be prepared by methods widely described in the literature, and can be conveniently protected.

Furthermore, some compounds of the present invention can also be obtained from other compounds of formula I by appropriate conversion reactions of functional groups in one or several steps, using well-known reactions in organic chemistry under the reported standard experimental conditions. Unless otherwise stated the meanings for the various substituents are as described in general formula I.

Thus, a group R² or R³ can be converted into another group R² or R³, giving rise to new compounds of formula I. For example, R²═H can be converted into R²=halogen, preferably bromo, by reaction with a suitable halogenating agent, in the same conditions described above for the preparation of compounds Ia;

or R²=halogen, preferably bromo, can be converted into R²=aryl or heteroaryl optionally substituted with one or more substituents selected from R^{c} and R^{g} by reaction with a boron derivative in the same conditions described above for the preparation of compounds Ib;

or R²=halogen, preferably bromo, can be converted into R²═NR^{b′}R^{c} by reaction with an amine of formula HNR^{b′}R^{c} in the same conditions described above for the preparation of compounds Ic;

or R²=halogen, preferably bromo, can be converted into R²=Cy¹ linked to the central ring through a N atom by reaction with the corresponding cyclic amine (i.e. Cy¹ wherein the N atom linked to the central ring is present as NH). This reaction is carried out under the same conditions described above for the preparation of compounds Ic;

or R² or R³=halogen can be converted into R² or R³═CN by reaction with a cyanide salt, such as CuCN, in a suitable solvent such as N-methylpyrrolidone and heating, preferably at reflux;

or R² or R³═NHR^{c′} can be converted into R²or R³═NCyR^{c′} by reaction with a compound of formula Cy-Y wherein Y represents halogen, preferably bromo, or —OSO₂CF₃ and wherein in R² Cy represents aryl or heteroaryl optionally substituted with one or more substituents selected from R^{c} and R^{g}; and in R³ Cy represents aryl or heteroaryl optionally substituted with one or more substituents selected from R^{b} and R^{h}. This reaction is carried out under the same conditions described above for the preparation of compounds Ic.

Other interconversions that can be performed upon R² and R³, as well as upon the substituents of R¹, to generate other compounds of formula I include, for example:

conversion of CN into CONH₂ by hydrolysis with a base such as KOH in a suitable solvent such as tert-butanol and heating, preferably to reflux;

conversion of CN into CH₂NH₂ by reaction with a reducing agent, such as LiAlH₄, in a suitable solvent such as diethyl ether or tetrahydrofuran;

conversion of a carboxylic acid group into an ester or an amide by reaction with an alcohol or an amine, respectively, in the presence of an activating agent such as N,N′-dicyclohexylcarbodiimide and 1-hydroxybenzotriazole and in a suitable solvent such as dimethylformamide; or alternatively a carboxylic acid can be converted into an acyl chloride by using standard conditions in organic synthesis and the acyl chloride can then be converted into an ester or amide by reaction with an alcohol or an amine respectively, in the presence of a base such as triethylamine, in a suitable solvent such as for example dichloromethane, and cooling, preferably at 0° C.;

conversion of an ester group into a carboxylic acid by hydrolysis, for example in the presence of a base, such as KOH, in a suitable solvent such as ethanol;

alkylation of an alcohol, thiol or amine by reaction with the corresponding alkylating agent such as a halide, preferably a chloride or bromide, in the presence of a base such as triethylamine, sodium hydroxide, sodium carbonate, potassium carbonate or sodium hydride, among others, in a suitable solvent such as dichloromethane, chloroform, dimethylformamide or toluene, and at a temperature comprised between room temperature and the temperature of the boiling point of the solvent;

conversion of an amine into an amide by reaction with a carboxylic acid, in the presence of a suitable condensing agent such as dicyclohexylcarbodiimide or N-(3-dimethylaminopropyl)-N′-ethylcarbodiimide hydrochloride in the presence of N-methylmorpholine optionally in the presence of 1-hydroxybenzotriazole, in a suitable solvent, such as dimethylformamide; or alternatively an amine can be converted into an amide by reaction with an acyl chloride in the same conditions mentioned above for the conversion of a carboxylic acid into an amide;

conversion of an amine into a urea or a carbamate by a two step sequence that comprises converting the amine into the corresponding isocyanate with triphosgene in the presence of a base such as diisopropylethylamine, triethylamine or N-methylmorpholine, in a suitable solvent such as acetonitrile or a halogenated hydrocarbon such as chloroform or dichloromethane, and then reacting the resulting isocyanate with the second amine in the case of the urea or with an alcohol in the case of the carbamate, in a suitable solvent, such as the solvent used in the first step; alternatively an amine can be converted into a urea or carbamate by reaction with an isocyanate or a chloroformate, respectively, in a suitable solvent, such as dimethylformamide, and at a suitable temperature, preferably room temperature;

conversion of an amine into a sulfonamide by reaction with a sulfonyl halide, such as sulfonyl chloride, optionally in the presence of catalytic amounts of a base such as 4-dimethylaminopyridine, in a suitable solvent such as dioxane, chloroform, dichloromethane or pyridine;

conversion of a sulfanyl group into a sulfinyl or sulfonyl group by reaction with 1 or 2 equivalents respectively of a suitable oxidizing agent, such as m-chloroperbenzoic acid, in a suitable solvent, such as dichloromethane;

conversion of a primary or secondary hydroxyl group into a leaving group, for example an alkylsulfonate or arylsulfonate such as mesylate or tosylate or a halogen such as Cl, Br or I, by reaction with a sulfonyl halide such as methanesulfonyl chloride, in the presence of a base, such as pyridine or triethylamine, in a suitable solvent such as dichloromethane or chloroform, or by reaction with a halogenating agent, such as SOCl₂, in a suitable solvent such as tetrahydrofuran; and said leaving group can then be substituted by reaction with an alcohol, amine or thiol, optionally in the presence of a base, such as K₂CO₃, NaH or KOH, and in a suitable solvent such as dimethylformamide, 1,2-dimethoxyethane or acetonitrile;

conversion of a CHO group into an amine by reaction with an amine in the presence of a reducing agent such as sodium triacetoxyborohydride or sodium cyanoborohydride, in a suitable solvent such as for example 1,2-dichloroethane;

conversion of NH₂ into NHCHR′R′, by reaction with a compound of formula R′COR′, wherein R′ represents H, C₁₋₄alkyl or Cy, in the same conditions described above for the conversion of a CHO group into an amine;

conversion of a NH₂ group into a 1,1-dioxothiomorpholine by reaction with vinyl sulfone, in an acid such as H₃PO₄ and preferably heating;

conversion of an ester group into an alcohol by reaction with a reducing agent, such as LiAlH₄, in a suitable solvent, such as tetrahydrofuran or diethyl ether;

conversion of a CHO group into an alcohol by reaction with a reducing agent, such as for example sodium borohydride, in a suitable solvent, such as for example tetrahydrofuran or methanol and at a suitable temperature, preferably room temperature;

conversion of an alcohol into a CHO group by reaction with a oxidizing agent, such as for example oxalyl chloride-dimethylsulfoxide in a suitable solvent such as for example dichloromethane, and cooling, preferably between −50 and −60° C.; or

conversion of a carboxylic acid into an amine by reaction with diphenylphosphorylazide, in the presence of a base, such as for example triethylamine, in a suitable solvent, such as dimethylformamide and at a suitable temperature, preferably 100° C., followed by aqueous treatment.

Some of these interconversion reactions are explained in greater detail in the examples.

As it will be obvious to those skilled in the art, these interconversion reactions can be carried out upon the compounds of formula I as well as upon any suitable synthesis intermediate thereof.

As mentioned previously, the compounds of the present invention act as p38 kinase inhibitors, inducing the reduction of proinflammatory cytokines. Therefore, the compounds of the invention are expected to be useful to treat or prevent diseases in which p38 plays a role in mammals, including human beings. This includes diseases caused by overproduction of cytokines such as TNF-α, IL-1, IL-6 or IL-8. These diseases include, but are not limited to, immune, autoimmune and inflammatory diseases, cardiovascular diseases, infectious diseases, bone resorption disorders, neurodegenerative diseases, proliferative diseases and processes associated with cyclooxygenase-2 induction. Preferred diseases to be treated or prevented with the compounds of the invention are immune, autoimmune and inflammatory diseases.

As an example, immune, autoimmune and inflammatory diseases that can be treated or prevented with the compounds of the present invention include rheumatic diseases (e.g. rheumatoid arthritis, psoriatic arthritis, infectious arthritis, progressive chronic arthritis, deforming arthritis, osteoarthritis, traumatic arthritis, gouty arthritis, Reiter's syndrome, polychondritis, acute synovitis and spondylitis), glomerulonephritis (with or without nephrotic syndrome), autoimmune hematologic disorders (e.g. hemolytic anemia, aplasic anemia, idiopathic thrombocytopenia and neutropenia), autoimmune gastritis and autoimmune inflammatory bowel diseases (e.g. ulcerative colitis and Crohn's disease), host versus graft disease, allograft rejection, chronic thyroiditis, Graves' disease, schleroderma, diabetes (type I and type II), active hepatitis (acute and chronic), primary biliary cirrhosis, myasthenia gravis, multiple sclerosis, systemic lupus erythematosus, psoriasis, atopic dermatitis, contact dermatitis, eczema, skin sunburns, chronic renal insufficiency, Stevens-Johnson syndrome, idiopathic sprue, sarcoidosis, Guillain-Barré syndrome, uveitis, conjunctivitis, keratoconjunctivitis, otitis media, periodontal disease, pulmonary interstitial fibrosis, asthma, bronchitis, rhinitis, sinusitis, pneumoconiosis, pulmonary insufficiency syndrome, pulmonary emphysema, pulmonary fibrosis, silicosis, chronic inflammatory pulmonary disease (e.g. chronic obstructive pulmonary disease) and other inflammatory or obstructive diseases of the airways.

Cardiovascular diseases that can be treated or prevented include, among others, myocardial infarction, cardiac hypertrophy, cardiac insufficiency, ischaemia-reperfusion disorders, thrombosis, thrombin-induced platelet aggregation, acute coronary syndromes, atherosclerosis and cerebrovascular accidents.

Infectious diseases that can be treated or prevented include, among others, sepsis, septic shock, endotoxic shock, sepsis by Gram-negative bacteria, shigellosis, meningitis, cerebral malaria, pneumonia, tuberculosis, viral myocarditis, viral hepatitis (hepatitis A, hepatitis B and hepatitis C), HIV infection, retinitis caused by cytomegalovirus, influenza, herpes, treatment of infections associated with severe burns, myalgias caused by infections, cachexia secondary to infections, and veterinary viral infections such as lentivirus, caprine arthritic virus, visna-maedi virus, feline immunodeficiency virus, bovine immunodeficiency virus or canine immunodeficiency virus.

Bone resorption disorders that can be treated or prevented include osteoporosis, osteoarthritis, traumatic arthritis and gouty arthritis, as well as bone disorders related with multiple myeloma, bone fracture and bone grafting and, in general, all these processes wherein it is necessary to induce osteoblastic activity and increase bone mass.

Neurodegenerative diseases that can be treated or prevented include Alzheimer's disease, Parkinson's disease, cerebral ischaemia and traumatic neurodegenerative disease, among others.

Proliferative diseases that can be treated or prevented include endometriosis, solid tumors, acute and chronic myeloid leukemia, Kaposi sarcoma, multiple myeloma, metastatic melanoma and angiogenic disorders such as ocular neovascularisation and infantile haemangioma.

p38 kinase inhibitors also inhibit the expression of proinflammatory proteins such as cyclooxygenase-2 (COX-2), the enzyme responsible for prostaglandin production. Therefore, the compounds of the present invention can also be used to treat or prevent diseases mediated by COX-2 and especially to treat processes with edema, fever and neuromuscular pain such as cephalea, pain caused by cancer, tooth pain, arthritic pain, hyperalgesia and allodynia.

In vitro and in vivo assays to determine the ability of a compound to inhibit p38 activity are well known in the art. For example, a compound to be tested can be contacted with the purified p38 enzyme to determine whether inhibition of p38 activity occurs. Alternatively, cell-based assays can be used to measure the ability of a compound to inhibit the production of cytokines such as TNFalpha, e.g. in stimulated peripheral blood mononuclear cells (PBMCs) or other cell types. Detailed disclosure of assays that can be used to test the biological activity of the compounds of the invention as p38 inhibitors can be found below (see Tests 1-3).

For selecting active compounds, testing at 10 μM must result in an activity of more than 50% inhibition in at least one of the tests mentioned above. More preferably, compounds should exhibit more than 50% inhibition at 1 μM, and still more preferably, they should exhibit more than 50% inhibition at 0.1 μM.

The present invention also relates to a pharmaceutical composition which comprises a compound of the present invention (or a pharmaceutically acceptable salt or solvate thereof) and one or more pharmaceutically acceptable excipients. The excipients must be “acceptable” in the sense of being compatible with the other ingredients of the composition and not deleterious to the recipients thereof.

The compounds of the present invention can be administered in the form of any pharmaceutical formulation, the nature of which, as it is well known, will depend upon the nature of the active compound and its route of administration. Any route of administration may be used, for example oral, parenteral, nasal, ocular, rectal and topical administration.

Solid compositions for oral administration include tablets, granulates and capsules. In any case the manufacturing method is based on a simple mixture, dry granulation or wet granulation of the active compound with excipients. These excipients can be, for example, diluents such as lactose, microcrystalline cellulose, mannitol or calcium hydrogenphosphate; binding agents such as for example starch, gelatin or povidone; disintegrants such as sodium carboxymethyl starch or sodium croscarmellose; and lubricating agents such as for example magnesium stearate, stearic acid or talc. Tablets can be additionally coated with suitable excipients by using known techniques with the purpose of delaying their disintegration and absorption in the gastrointestinal tract and thereby provide a sustained action over a longer period, or simply to improve their organoleptic properties or their stability. The active compound can also be incorporated by coating onto inert pellets using natural or synthetic film-coating agents. Soft gelatin capsules are also possible, in which the active compound is mixed with water or an oily medium, for example coconut oil, mineral oil or olive oil.

Powders and granulates for the preparation of oral suspensions by the additon of water can be obtained by mixing the active compound with dispersing or wetting agents; suspending agents and preservatives. Other excipients can also be added, for example sweetening, flavouring and colouring agents.

Liquid forms for oral administration include emulsions, solutions, suspensions, syrups and elixirs containing commonly-used inert diluents, such as purified water, ethanol, sorbitol, glycerol, polyethylene glycols (macrogols) and propylene glycol. Said compositions can also contain coadjuvants such as wetting, suspending, sweetening, flavouring agents, preservatives and buffers.

Injectable preparations, according to the present invention, for parenteral administration, comprise sterile solutions, suspensions or emulsions, in an aqueous or non-aqueous solvent such as propylene glycol, polyethylene glycol or vegetable oils. These compositions can also contain coadjuvants, such as wetting, emulsifying, dispersing agents and preservatives. They may be sterilized by any known method or prepared as sterile solid compositions which will be dissolved in water or any other sterile injectable medium immediately before use. It is also possible to start from sterile materials and keep them under these conditions throughout all the manufacturing process.

For the rectal administration, the active compound can be preferably formulated as a suppository on an oily base, such as for example vegetable oils or solid semisynthetic glycerides, or on a hydrophilic base such as polyethylene glycols (macrogol).

The compounds of the invention can also be formulated for their topical application for the treatment of pathologies occurring in zones or organs accessible through this route, such as eyes, skin and the intestinal tract. Formulations include creams, lotions, gels, powders, solutions and patches wherein the compound is dispersed or dissolved in suitable excipients.

For the nasal administration or for inhalation, the compound can be formulated as an aerosol and it can be conveniently released using suitable propellants.

The dosage and frequency of doses will depend upon the nature and severity of the disease to be treated, the age, the general condition and body weight of the patient, as well as the particular compound administered and the route of administration, among other factors. A representative example of a suitable dosage range is from about 0.01 mg/Kg to about 100 mg/Kg per day, which can be administered as a single or divided doses.

The activity of the compounds of this invention can be assessed using the following tests:

### Test 1: Inhibition of TNF-α Release Induced by LPS in Human Peripheral Blood Mononuclear Cells

To obtain the mononuclear cells: heparinized venous blood, obtained from healthy volunteers, is diluted with an equal volume of saline phosphate buffer without calcium or magnesium. Aliquots of 30 mL of the mixture are transferred to 50 mL centrifuge tubes containing 15 mL of Ficoll-Hypaque (1.077 g/mL). The tubes are centrifuged at 1200×g for 20 min at room temperature without braking. Approximately two-thirds of the band of platelets lying above the mononuclear cells is removed with a pipette. The mononuclear cells are carefully transferred to a 50 mL tube, washed twice with saline phosphate buffer, centrifuged at 300×g for 10 min at room temperature and resuspended in RPMI supplemented with 1% inactivated fetal bovine serum at a cell density of 2×10⁶ cells/mL.

Inhibition of TNF-α release: 100 μL of mononuclear cells (2×10⁶ cells/mL) are incubated on 96-well plates with 50 μL of the test product (final concentration, 0.001-10 μM) and 50 μL LPS (E. coli 055B5, Sigma) at a final concentration of 400 ng/mL for 19 h at 37° C. in an atmosphere with CO₂ at 5%. The amount of TNF-α released in the supernatant is quantified using a commercial ELISA kit (Biosource International).

### Test 2: Inhibition of TNF-α Release Induced by LPS in Human Histiocytic Lymphoma Cells, U-937

Maintenance and differentiation of U-937 cells: U-937 cells (ATCC N^{o} CRL-159.2) are cultivated in RPMI 1640 medium supplemented with 10% inactivated fetal bovine serum (Gibco). A total of 0.5×10⁶ cells are incubated in the presence of 20 ng/mL of PMA (phorbol 12-myristate 13-acetate) for 24 h to achieve complete monocytic differentiation. All the incubations are carried out at 37° C. in an atmosphere with 5% CO₂. The cells are centrifuged (200×g for 5 min) and resuspended in RPMI 1640 medium supplemented with 2% inactivated fetal bovine serum at a density of 2×10⁶ cells/mL.

Inhibition of TNF-α release: 100 μL of cells U-937 (2×10⁶ cells/mL) are incubated with 100 μL of the test product (final concentration, 0.001-10 μM) for 30 min in 96-well plates. The mother solutions of the products (10 mM in DMSO) are diluted in culture medium to reach a final DMSO concentration equal to or less than 0.1%. A total of 20 μL of LPS (E. coli 055B5, Sigma) are added to a final concentration of 100 ng/mL and after incubation for 4 hours the amount of TNF-α released in the supernatant is quantified using a commercial ELISA kit (Biosource International).

Test 3: Inhibition of p38-α Kinase:

In a final volume of 25 μL, a total of 5 μL of the test product (final concentration, 0.001-10 μM), 5-10 mU of p38-α with 0.33 mg/mL of myelin basic protein, Mg²⁺ acetate (10 mM) and [γ³³P-ATP] (100 μM, specific activity 500 cpm/pmol) in buffer Tris 25 mM pH7.5, EGTA 0.02 mM is incubated. The reaction is started by adding Mg²⁺[γ³³P-ATP]. After incubation for 40 min at room temperature, the reaction is quenched by adding 5 μL of 3% phosphoric acid solution. The reaction mixture (10 μL) is passed through a filter (P30) and washed three times for 5 min with a 75 mM phosphoric acid solution and once with methanol before drying it and counting it, by liquid scintillation.

Compounds of all examples exhibited more than 50% inhibition at 10 μM in at least one of the above assays. When tested at 1 μM, compounds of examples 1-15 and 17-36 exhibited more than 50% inhibition in at least one of the above assays.

The invention is illustrated by the following examples.

### EXAMPLES

The following abbreviations have been used in the examples:
ACN: acetonitrile
Ac₂O: acetic anhydride
^{t}BuONa: sodium tert-butoxide
DMF: dimethylformamide
DMSO: dimethylsulfoxide
EtOAc: ethyl acetate
EtOH: ethanol
HOAc: acetic acid
MeOH: methanol
NH₄OAc: ammonium acetate
TEA: triethylamine
THF: tetrahydrofuran
t_{R}: retention time
LC-MS: liquid chromatography-mass spectrometry
LC-MS spectra have been performed using the following chromatographic methods:
Method 1: Column Tracer Excel 120, ODSB 5 μm (10 mm×0.21 mm), temperature: 30° C., flow: 0.35 mL/min, eluent: A=ACN, B=0.1% HCOOH, gradient: 0 min 10% A −10 min 90% A.
Method 2: Column Tracer Excel 120, ODSB 5 μm (10 mm×0.21 mm), temperature: 30° C., flow: 0.40 mL/min, eluent: A=ACN, B=0.1% HCOOH, isocratic: 43% A.
Method 3: Column X-Terra MS C18 5 μm (150 mm×2.1 mm), temperature: 30° C., flow: 0.40 mL/min, eluent: A=ACN, B=10 mM NH₄OAc (pH =6.80), gradient: 0 min 25% A −6 min 80% A −7.5 min 25% A.

The MS spectra have been obtained with positive electrospray ionization mode over a scan range from 100 to 800 amu.

### REFERENCE EXAMPLE 1

### 5,6,7,8-Tetrahydroisoquinoline N-oxide

To a solution of 5,6,7,8-tetrahydroisoquinoline (30.00 g, 0.22 mol) in HOAc (135 mL), H₂O₂ at 30% (34 mL, 0.33 mol) was added. It was heated at 80° C. overnight and allowed to cool to room temperature. The solvent was concentrated to a quarter of the initial volume and water (110 mL) was added. The solvent was concentrated to a quarter of the initial volume and CHCl₃ and water were added. The pH was adjusted to 7 with solid K₂CO₃ and the phases were separated. The aqueous phase was extracted 3 times with CHCl₃. The combined organic phases were dried over Na₂SO₄ and the solvent was evaporated, to afford the title compound as a yellow solid (yield: quantitative).

¹H NMR (300 MHz, CDCl₃) δ (TMS): 1.81 (m, 4 H), 2.71 (m, 4 H), 6.98 (d, J=6.6 Hz, 1 H), 7.97 (d, J=6.6 Hz, 1 H), 8.00 (s, 1 H).

### REFERENCE EXAMPLE 2

### 5,6,7,8-Tetrahydroisoquinolin-5-ol

A solution of 5,6,7,8-tetrahydroisoquinoline N-oxide (56.00 g, 0.38 mol, obtained in reference example 1) in Ac₂O (100 mL) was added dropwise over Ac₂O (76 mL) previously heated to reflux. Once the addition was finished, it was stirred at reflux for 90 min and allowed to cool to room temperature. The solvent was evaporated. CHCl₃ and water were added and the phases were separated. The aqueous phase was reextracted 2 times with CHCl₃. The combined organic phases were dried over Na₂SO₄ and the solvent was evaporated. 12% HCl (375 mL) was added to the obtained residue and it was heated at reflux for 5 h. It was allowed to cool and the pH was adjusted at 7 with 2 N NaOH. The aqueous phase was extracted with CHCl₃. The combined organic phases were dried over Na₂SO₄ and the solvent was evaporated. The crude product obtained was purified by chromatography on silica gel using hexane-EtOAc mixtures of increasing polarity as eluent, to afford 18.94 g of the desired compound (yield: 34%).

¹H NMR (300 MHz, CDCl₃) δ (TMS): 1.79 (m, 2 H), 1.90-2.16 (complex signal, 2 H), 2.76 (m, 2 H), 3.30 (broad s, 1 H, OH), 4.74 (m, 1 H), 7.38 (d, J=5.1 Hz, 1 H), 8.33 (s, 1 H), 8.37 (d, J=5.1 Hz, 1 H).

### REFERENCE EXAMPLE 3

### 7,8-Dihydro-6H-isoquinolin-5-one

To a solution of oxalyl chloride (1.9 mL, 22 mmol) in CH₂Cl₂ (80 mL) under argon and previously cooled at −50/−60° C., a solution of DMSO (3.1 mL, 44 mmol) in CH₂Cl₂ (10 mL) was added. It was stirred for 5 min at −50/−60° C. and a solution of 5,6,7,8-tetrahydroisoquinolin-5-ol (3.00 g, 20 mmol, obtained in reference example 2) in CH₂Cl₂ (20 mL) was added. After stirring for 15 min at −50/−60° C., TEA (13.9 mL, 100 mmol) was added. It was stirred for 5 min at −50/−60° C. and allowed to warm to room temperature. It was poured over a mixture of water-ice. It was extracted 3 times with CH₂Cl₂. The combined organic phases were dried over Na₂SO₄ and the solvent was evaporated. The crude product obtained was purified by chromatography on silica gel using hexane-EtOAc mixtures of increasing polarity as eluent, to afford 2.63 g of the desired compound as an orange liquid (yield: 89%).

LC-MS (method 1): t_{R}=2.52 min; m/z=148.0 [M+H]⁺.

### REFERENCE EXAMPLE 4

### Methyl (5-oxo-5,6,7,8-tetrahydroisoquinolin-6-yl)oxoacetate

To a solution of 7,8-dihydro-6H-isoquinolin-5-one (1.61 g, 11 mmol, obtained in reference example 3) in 1,2-dimethoxyethane (130 mL) under argon, a solution of ethyl oxalate (3.20 g, 22 mmol) in 1,2-dimethoxyethane (12 mL) was added. Then, sodium methoxide (25% in MeOH, 5.0 mL, 22 mmol) was added dropwise and it was stirred at room temperature overnight. It was poured over a mixture of water-ice. The phases were separated. The aqueous phase was extracted 2 times with CHCl₃. The organic phases were discarded. The aqueous phase was acidified with HOAc to pH 5 and extracted 3 times with CHCl₃. The combined organic phases were dried over Na₂SO₄ and the solvent was evaporated, to afford 2.55 g of the title compound (yield: quantitative).

LC-MS (method 1): t_{R}=3.08 min; m/z=234.1 [M+H]⁺.

### REFERENCE EXAMPLE 5

### 5-Oxo-5,6,7,8-tetrahydroisoquinolin-6-carbaldehyde

Following a similar procedure to that described in reference example 4, but starting from ethyl formate instead of ethyl oxalate, the title compound was obtained.

LC-MS (method 1): t_{R}=3.85 min (flow =0.30 mL/min); m/z=176.2 [M+H]⁺.

### REFERENCE EXAMPLE 6

### 6-(2,2-Diethoxyacetyl)-7,8-dihydro-6H-isoquinolin-5-one

Following a similar procedure to that described in reference example 4, but starting from ethyl diethoxyacetate instead of ethyl oxalate, the title compound was obtained.

¹H NMR (300 MHz, CDCl₃) δ (TMS): 1.27 (m, 6 H), 1.59 (broad s, OH+H₂O), 2.84 (s, 4 H), 3.63 (m, 2 H), 3.74 (m, 2 H), 5.04 (s, 1 H), 7.69 (d, J=5.0 Hz, 1 H), 8.54 (broad s, 1 H), 8.62 (d, J=5.0 Hz, 1 H).

### REFERENCE EXAMPLE 7

### Methyl 1-(4-fluorophenyl)-4,5-dihydropyrazolo[3,4-f]isoquinolin-3-carboxylate

To a solution of methyl (5-oxo-5,6,7,8-tetrahydroisoquinolin-6-yl)oxoacetate (327 mg, 1.4 mmol, obtained in reference example 4) in 2,2,2-trifluoroethanol (14 mL), 4-fluorophenylhydrazine hydrochloride (228 mg, 1.4 mmol) and 95-97% H₂SO₄ (3 drops) were added. The mixture was heated at reflux overnight. It was allowed to cool and CHCl₃ and saturated NaHCO₃ solution were added. The aqueous phase was extracted 3 times with CHCl₃. The combined organic phases were dried over Na₂SO₄ and the solvent was evaporated. The crude product obtained was purified by chromatography on silica gel using hexane-EtOAc mixtures of increasing polarity as eluent, to afford 297 mg of the desired compound (yield: 65%).

LC-MS (method 1): t_{R}=5.12 min; m/z=324.1 [M+H]⁺.

### REFERENCE EXAMPLES 8-9

Following a similar procedure to that described in reference example 7, but starting from the appropriate compounds in each case, the compounds in the following table were obtained:

| | | | LC-MS | | |
|---|---|---|---|---|---|
| Reference | | | | t_{R} | m/z |
| example | Compound name | Starting products | Method | (min) | [M + H]⁺ |
| 8 | 1-(4-Fluorophenyl)-4,5- | Reference example 5 | 1 | 4.47 | 266.1 |
| | dihydropyrazolo[3,4- | and 4-fluorophenyl- | | | |
| | f]isoquinoline | hydrazine | | | |
| | | hydrochloride | | | |
| 9 | 1-(3-Trifluoromethylphenyl)- | Reference example 5 | 1 | 5.78 | 316.2 |
| | 4,5-dihydropyrazolo[3,4- | and (3-trifluoromethyl- | | | |
| | f]isoquinoline | phenyl)hydrazine | | | |

### REFERENCE EXAMPLE 10

### 3-Diethoxymethyl-1-(4-fluorophenyl)-4,5-dihydropyrazolo[3,4-f]isoquinoline

Following a similar procedure to that described in reference example 7, but starting from 6-(2,2-diethoxyacetyl)-7,8-dihydro-6H-isoquinolin-5-one (obtained in reference example 6) and 4-fluorophenylhydrazine hydrochloride, the title compound was obtained.

¹H NMR (300 MHz, CDCl₃) δ (TMS): 1.27 (m, 6 H), 2.97 (broad s, 4 H), 3.64 (m, 2 H), 3.76 (m, 2 H), 5.63 (s, 1 H), 6.58 (d, J=5.4 Hz, 1 H), 7.19 (t, J=8.4 Hz, 2 H), 7.46 (m, 2 H), 8.26 (d, J=5.4 Hz, 1 H), 8.52 (s, 1 H).

### REFERENCE EXAMPLE 11

### 1-(4-Fluorophenyl)-4,5-dihydropyrazolo[3,4-f]isoquinolin-3-carbaldehyde

A solution of 3-diethoxymethyl-1-(4-fluorophenyl)-4,5-dihydropyrazolo[3,4-f]isoquinoline (0.62 g, 1.7 mmol, obtained in reference example 10) in 1 N HCl (7 mL) was heated at 80-90° C. for 1 h. It was allowed to cool and pH was adjusted to 7 with 2 N NaOH. It was diluted with CH₂Cl₂ and saturated NaHCO₃ solution was added. The phases were separated and the aqueous phase was reextracted 2 times with CH₂Cl₂. The combined organic phases were washed with brine, dried over Na₂SO₄ and the solvent was evaporated, to afford 0.51 g of the title compound as a yellow solid (yield: quantitative).

¹H NMR (300 MHz, CDCl₃) δ (TMS): 3.01 (m, 2 H), 3.19 (m, 2 H), 6.59 (d, J=5.1 Hz, 1 H), 7.27 (m, 2 H), 7.53 (m, 2 H), 8.31 (d, J=5.1 Hz, 1 H), 8.57 (s, 1 H), 10.12(s,1 H).

### EXAMPLE 1

### 5-Bromo-1-(4-fluorophenyl)pyrazolo[3,4-f]isoquinoline

To a solution of 1-(4-fluorophenyl)-4,5-dihydropyrazolo[3,4-f]isoquinoline (1.45 g, 5.5 mmol, obtained in reference example 8) in CHCl₃ (30 mL) and CCl₄ (50 mL), N-bromosuccinimide (4.88 g, 27.4 mmol) and 2,2′-azobis(2-methylbutyronitrile) (60 mg) were added and it was heated at 70-75° C. for 3.5 h. It was allowed to cool and the solvent was evaporated. The residue was dissolved in CHCl₃ and washed 3 times with 0.5 N NaOH. The basic aqueous phase was reextracted 2 times with CHCl₃. The combined organic phases were washed with brine, dried over Na₂SO₄ and the solvent was evaporated. The crude product obtained was purified by chromatography on silica gel using hexane-EtOAc mixtures of increasing polarity as eluent, to afford 1.1 g of the desired compound as a solid (yield: 58%).

LC-MS (method 1): t_{R}=8.96 min; m/z=342.0, 344.0 [M+H]⁺.

### EXAMPLE 2

### 1-(4-Fluorophenyl)-5-[4-(tetrahydropyran-2-yloxy)phenyl]pyrazolo[3,4-f]isoquinoline

To a suspension of 5-bromo-1-(4-fluorophenyl)pyrazolo[3,4-f]isoquinoline (0.2 g, 0.6 mmol, obtained in example 1), [4-(tetrahydropyran-2-yloxy)phenyl]boronic acid (0.19 g, 0.9 mmol), anhydrous K₂CO₃ (0.16 g, 1.2 mmol), Pd(PPh₃)₄ (4.7 mg) in 1,2-dimethoxyethane (4 mL) under argon, water (0.12 mL) was added. The reaction mixture was heated at 80° C. overnight. It was allowed to cool and water and CH₂Cl₂ were added. The phases were separated and the aqueous phase was reextracted 3 times with CH₂Cl₂. The combined organic phases were dried over Na₂SO₄ and the solvent was evaporated. The crude product obtained was purified by chromatography on silica gel using hexane-EtOAc mixtures of increasing polarity as eluent, to afford 0.25 g of the title compound (yield: 97%).

LC-MS (method 1): t_{R}=10.10 min; m/z=440.1 [M+H]⁺.

### EXAMPLES 3-7

Following a similar procedure to that described in example 2, but starting from the appropriate compounds in each case, the compounds in the following table were obtained:

| | | | LC-MS | | |
|---|---|---|---|---|---|
| | | | | t_{R} | m/z |
| Example | Compound name | Starting products | Method | (min) | [M + H]⁺ |
| 3 | 1-(4-Fluorophenyl)-5-(4- | Example 1 and (4- | 1 | 5.32 | 341.0 |
| | pyridyl)pyrazolo[3,4-f]isoquinoline | pyridyl)boronic acid | | | |
| 4 | 1-(4-Fluorophenyl)-5-phenyl- | Example 1 and | 2 | 5.66 | 340.0 |
| | pyrazolo[3,4-f]isoquinoline | phenylboronic acid | | | |
| 5 | 5-(2-Chlorophenyl)-1-(4- | Example 1 and (2- | 1 | 9.54 | 374.0 |
| | fluorophenyl)pyrazolo[3,4- | chlorophenyl)boronic | | | 375.9 |
| | f]isoquinoline | acid | | | |
| 6 | 1-(4-Fluorophenyl)-5-(3-pyridyl)- | Example 1 and (3- | 1 | 5.64 | 341.0 |
| | pyrazolo[3,4-f]isoquinoline | pyridyl)boronic acid | | | |
| 7 | 5-(4-Aminophenyl)-1-(4- | Example 1 and (4- | 1 | 5.84 | 355.0 |
| | fluorophenyl)pyrazolo[3,4- | aminophenyl)boronic | | | |
| | f]isoquinoline | acid | | | |

### EXAMPLE 8

### [1-(4-Fluorophenyl)-5-(3-pyridyl)pyrazolo[3,4-f]isoquinolin-3-yl]methanol

### a) Methyl 5-bromo-1-(4-fluorophenyl)pyrazolo[3,4-f]isoquinolin-3-carboxylate

To a solution of methyl 1-(4-fluorophenyl)-4,5-dihydropyrazolo[3,4-f]isoquinolin-3-carboxylate (200 mg, 0.6 mmol, obtained in reference example 7) in trimethyl phosphate (2.2 mL), it was successively added phosphorus pentoxide (474 mg, 3.3 mmol) and dropwise a solution of bromine (0.25 mL) in trimethyl phosphate (1 mL). The mixture was heated at 60° C. for 6 h and allowed to cool to room temperature. EtOAc (50 mL), water (50 mL) and conc. (30%) NH₃ were added until pH 8-9. The phases were separated. The aqueous phase was reextracted 3 times with EtOAc. The combined organic phases were extracted 3 times with 6 N HCl. The combined acidic aqueous phases were basified with conc. (10%) NH₃ until pH 9 and extracted 3 times with EtOAc. The combined organic phases were dried over Na₂SO₄ and the solvent was evaporated. The crude product obtained was purified by chromatography on silica gel using hexane-EtOAc mixtures of increasing polarity as eluent, to afford 150 mg of the desired compound (yield: 60%).

LC-MS (method 1): t_{R}=9.58 min; m/z=399.9, 401.9 [M+H]⁺.

### b) Methyl 1-(4-fluorophenyl)-5-(3-pyridyl)pyrazolo[3,4-f]isoquinolin-3-carboxylate

Following a similar procedure to that described in example 2, but starting from methyl 5-bromo-1-(4-fluorophenyl)pyrazolo[3,4-f]isoquinolin-3-carboxylate (obtained in section a) and (3-pyridyl)boronic acid, the desired compound was obtained.

LC-MS (method 1): t_{R}=6.61 min; m/z=399.1 [M+H]⁺.

### c) Title Compound

To a suspension of LiAlH₄ (18 mg, 0.5 mmol) in THF (1.0 mL) under argon and cooled at 0° C., a suspension of methyl 1-(4-fluorophenyl)-5-(3-pyridyl)pyrazolo[3,4-f]isoquinolin-3-carboxylate (100 mg, 0.2 mmol, obtained in section b) in THF (2 mL) was added dropwise. The mixture was stirred at 0° C. for 30 min, allowed to warm up to room temperature and stirred at this temperature overnight. It was cooled to 0° C. and a mixture of water (0.15 mL) and THF (0.30 mL), 4 N NaOH (0.15 mL) and water (0.35 mL) were succesively added dropwise. It was stirred at room temperature for 30 min. It was filtered through celite and washed with CHCl₃. Then, CHCl₃ and water were added to the filtrate and the phases were separated. The aqueous phase was extracted 2 times with CHCl₃. The combined organic phases were dried over Na₂SO₄ and the solvent was evaporated. The crude product obtained was purified by chromatography on silica gel using hexane-EtOAc mixtures of increasing polarity as eluent, to afford 47 mg of the title compound (yield: 51%).

LC-MS (method 1): t_{R}=4.67 min; m/z=371.0 [M+H]⁺.

### EXAMPLE 9

### [1-(4-Fluorophenyl)-5-phenylpyrazolo[3,4-f]isoquinolin-3-yl]methanol

### a) Methyl 1-(4-fluorophenyl)-5-phenylpyrazolo[3,4-f]isoquinolin-3-carboxylate

Following a similar procedure to that described in example 2, but starting from methyl 5-bromo-1-(4-fluorophenyl)pyrazolo[3,4-f]isoquinolin-3-carboxylate (obtained in example 8 section a) and phenylboronic acid, the desired compound was obtained.

¹ H NMR (300 MHz, CDCl₃) δ (TMS): 4.08 (s, 3 H), 7.34-7.40 (complex signal, 3 H), 7.55 (m, 5 H), 7.62-7.67 (complex signal, 2 H), 8.36 (s, 1 H), 8.53 (d, J=6.0 Hz, 1 H), 9.37 (s, 1 H).

### b) Title Compound

To a suspension of LiAlH₄ (90 mg, 2.3 mmol) in THF (3.5 mL) under argon and cooled at 0° C., a solution of methyl 1-(4-fluorophenyl)-5-phenylpyrazolo[3,4-f]isoquinolin-3-carboxylate (456 mg, 1.1 mmol, obtained in section a) in THF (8 mL) was added dropwise. The mixture was allowed to warm up to room temperature and stirred at this temperature for 2 h 45 min. It was cooled to 0° C. and a mixture of water (0.15 mL) and THF (0.30 mL), 4 N NaOH (0.15 mL) and water (0.35 mL) were succesively added dropwise. The mixture was stirred at room temperature for 30 min, was then filtered through celite and washed with CHCl₃. CHCl₃ and water were added to the filtrate and the phases were separated. The aqueous phase was extracted 2 times with CHCl₃. The combined organic phases were dried over Na₂SO₄ and the solvent was evaporated. The crude product obtained was purified by chromatography on silica gel using hexane-EtOAc mixtures of increasing polarity as eluent, to afford 166 mg of the title compound (yield: 39%).

LC-MS (method 1): t_{R}=6.83 min; m/z=370.1 [M+H]⁺.

### EXAMPLE 10

### 5-(3-Pyridyl)-1-(3-trifluoromethylphenyl)pyrazolo[3,4-f]isoquinoline

### a) 5-Bromo-1-(3-trifluoromethylphenyl)pyrazolo[3,4-f]isoquinoline

Following a similar procedure to that described in example 8 section a, but starting from 1-(3-trifluoromethylphenyl)-4,5-dihydropyrazolo[3,4-f]isoquinoline (obtained in reference example 9), the desired compound was obtained.

LC-MS (method 1): t_{R}=9.86 min; m/z=392.0, 394.0 [M+H]⁺.

### b) Title Compound

Following a similar procedure to that described in example 2, but starting from 5-bromo-1-(3-trifluoromethylphenyl)pyrazolo[3,4-f]isoquinoline (obtained in section a) and (3-pyridyl)boronic acid, using dioxane as solvent and carrying out the reaction at 95° C., the title compound was obtained.

LC-MS (method 1): t_{R}=6.85 min; m/z=391.1 [M+H]⁺.

### EXAMPLE 11

### 3-Bromo-1-(4-fluorophenyl)-5-(3-pyridyl)pyrazolo[3,4-f]isoquinoline

Following a similar procedure to that described in example 8 section a, but starting from 1-(4-fluorophenyl)-5-(3-pyridyl)pyrazolo[3,4-f]isoquinoline (obtained in example 6), the title compound was obtained.

LC-MS (method 1): t_{R}=8.12 min; m/z=419.1, 421.1 [M+H]⁺.

### EXAMPLE 12

### 3-Aminomethyl-1-(4-fluorophenyl)-5-phenylpyrazolo[3,4-f]isoquinoline

### a) 3-Chloromethyl-1-(4-fluorophenyl)-5-phenylpyrazolo[3,4-f]isoquinoline hydrochloride

To a suspension of [1-(4-fluorophenyl)-5-phenylpyrazolo[3,4-f]isoquinolin-3-yl]methanol (123 mg, 0.3 mmol, obtained in example 9) in THF (1.5 mL) under argon, a solution of SOCl₂ (112 mg, 0.9 mmol) in THF (0.5 mL) was added. The mixture was stirred at room temperature for 6 h 30 min. The solvent was evaporated to dryness and toluene was added to the obtained solid. The resulting suspension was filtered. The solid obtained was washed with toluene and dried under vacuum, to afford 152 mg of the desired compound as a solid (yield: quantitative).

LC-MS (method 1): t_{R}=10.22 min; m/z=388.0, 390.0 [M+H]⁺.

### b) 1-(4-Fluorophenyl)-5-phenyl-3-(phthalimidomethyl)pyrazolo[3,4-f]isoquinoline

To a suspension of 3-chloromethyl-1-(4-fluorophenyl)-5-phenylpyrazolo[3,4-f]isoquinoline hydrochloride (150 mg, 0.3 mmol, obtained in section a) in anhydrous DMF (4 mL) under argon, potassium phthalimide (190 mg, 1.0 mmol) was added and the resulting mixture was heated at 60° C. for 6 h. The solvent was evaporated and CHCl₃ and water were added. The phases were separated. The aqueous phase was reextracted 2 times with CHCl₃. The combined organic phases were washed 2 times with 1 N NaOH, dried over Na₂SO₄ and the solvent was evaporated. The crude product obtained was purified by chromatography on silica gel using hexane-EtOAc mixtures of increasing polarity as eluent, to afford 85 mg of the desired compound (yield: 52%).

LC-MS (method 1): t_{R}=10.08 min; m/z=499.1 [M+H]⁺.

### c) Title Compound

To a suspension of 1-(4-fluorophenyl)-5-phenyl-3-(phthalimidomethyl)pyrazolo[3,4-f]isoquinoline (85 mg, 0.2 mmol, obtained in section b) in EtOH (1.5 mL) under argon, a solution of hydrazine monohydrate (17 mg, 0.3 mmol) in EtOH (1 mL) was added, and the mixture was heated at reflux for 3 h. The solvent was evaporated and CHCl₃, water and 1 N NaOH were added. The phases were separated. The aqueous phase was reextracted 2 times with CHCl₃. The combined organic phases were dried over Na₂SO₄ and the solvent was evaporated. The crude product obtained was purified by chromatography on silica gel using EtOAc-MeOH—NH₃ mixtures of increasing polarity as eluent, to afford 42 mg of the title compound (yield: 67%).

LC-MS (method 1): t_{R}=4.90 min; m/z=369.1 [M+H]⁺.

### EXAMPLE 13

### 4-[1-(4-Fluorophenyl)pyrazolo[3,4-f]isoquinolin-5-yl]benzoic acid

### a) Methyl 4-[1-(4-fluorophenyl)pyrazolo[3,4-f]isoquinolin-5-yl]benzoate

Following a similar procedure to that described in example 2, but starting from 5-bromo-1-(4-fluorophenyl)pyrazolo[3,4-f]isoquinoline (obtained in example 1) and (4-methoxycarbonylphenyl)boronic acid, the desired compound was obtained.

¹H NMR (300 MHz, CDCl₃) δ (TMS): 4.00 (s, 3 H), 7.35 (t, J=5.9 Hz, 2 H), 7.43 (d, J=6.0 Hz, 1 H), 7.60-7.65 (complex signal, 4 H), 7.81 (s, 1H), 8.22 (d, J=8.4 Hz, 2 H), 8.33 (s, 1 H), 8.49 (d, J=5.7 Hz, 1 H), 9.25 (s, 1H).

### b) Title Compound

To a suspension of methyl 4-[1-(4-fluorophenyl)pyrazolo[3,4-f]isoquinolin-5-yl]benzoate (97 mg, 0.2 mmol, obtained in section a) in EtOH (6 mL), a solution of KOH (110 mg, 1.9 mmol) in water (0.8 mL) was added dropwise. The mixture was heated at reflux for 5 h and allowed to cool to room temperature. The solvent was evaporated, EtOAc and water were added and the phases were separated. The pH of the aqueous phase was adjusted to 6 with HOAc. The solid obtained was collected by filtration, to afford 72 mg of the title compound (yield: 77%).

LC-MS (method 1): t_{R}=7.00 min; m/z=384.1 [M+H]⁺.

### EXAMPLE 14

### 3-Amino-1-(4-fluorophenyl)-5-(3-pyridyl)pyrazolo[3,4-f]isoquinoline

### a) 1-(4-Fluorophenyl)-5-(3-pyridyl)pyrazolo[3,4-f]isoquinolin-3-carboxylic acid

Following a similar procedure to that described in example 13 section b, but starting from methyl 1-(4-fluorophenyl)-5-(3-pyridyl)pyrazolo[3,4-f]isoquinolin-3-carboxylate (obtained in example 8 section b), the desired compound was obtained.

LC-MS (method 1): t_{R}=4.96 min; m/z=385.1 [M+H]⁺

### b) Title Compound

To a suspension of 1-(4-fluorophenyl)-5-(3-pyridyl)pyrazolo[3,4-f]isoquinolin-3-carboxylic acid (0.43 g, 1.1 mmol, obtained in section a) in DMF (9 mL) under argon, a solution of TEA (170 mg, 1.7 mmol) in DMF (2 mL) and a solution of diphenylphosphorylazide (0.46 g, 1.7 mmol) in DMF (2 mL) were succesively added. The reaction mixture was stirred at room temperature for 2 h 30 min. Water (1.1 mL) was added dropwise and the reaction was heated to 100° C. It was stirred at this temperature for 1 h and allowed to cool to room temperature. The solvent was evaporated and CHCl₃ was added. The organic phase was washed 3 times with saturated NaHCO₃, dried over Na₂SO₄ and the solvent was evaporated. The crude product obtained was purified by chromatography on silica gel using hexane-EtOAc mixtures of increasing polarity as eluent, to afford 120 mg of the title compound (yield: 30%).

LC-MS (method 1): t_{R}=4.48 min; m/z=356.2 [M+H]⁺.

### EXAMPLE 15

### 1-(4-Fluorophenyl)pyrazolo[3,4-f]isoquinolin-5-carbonitrile

A suspension of 5-bromo-1-(4-fluorophenyl)pyrazolo[3,4-f]isoquinoline (210 mg, 0.6 mmol, obtained in example 1) and CuCN (110 mg, 1.2 mmol) in N-methyl-2-pyrrolidone (4 mL) under argon was heated at 200° C. for 2 h. The mixture was allowed to cool and poured into a solution of ethylenediamine at 10%. CHCl₃ was added and the phases were separated. The aqueous phase was reextracted 6 times with CHCl₃. The combined organic phases were dried over Na₂SO₄ and the solvent was evaporated. Remaining N-methyl-2-pyrrolidone was distilled off under vacuum. The crude product obtained was purified by chromatography on silica gel using hexane-EtOAc mixtures of increasing polarity as eluent, to afford 131 mg of the title compound (yield: 74%).

LC-MS (method 1): t_{R}=7.85 min; m/z=289.0 [M+H]⁺.

### EXAMPLE 16

### 5-Aminomethyl-1-(4-fluorophenyl)pyrazolo[3,4-f]isoquinoline

To a suspension of LiAlH₄ (14 mg, 0.3 mmol) in THF (1 mL) cooled at 0° C. under argon, a solution of 1-(4-fluorophenyl)pyrazolo[3,4-f]isoquinolin-5-carbonitrile (25 mg, 0.1 mmol, obtained in example 15) in THF (1 mL) was added dropwise. The mixture was allowed to warm up to room temperature and stirred at this temperature for 3 h. It was cooled to 0° C. and a mixture of water (0.02 mL) and THF (0.04 mL), 4 N NaOH (0.02 mL) and water (0.05 mL) were succesively added. It was stirred at room temperature for 30 min and filtered through celite. It was washed with CHCl₃. CHCl₃ and brine were added to the filtrate and the phases were separated. The aqueous phase was extracted 2 times with CHCl₃. The combined organic phases were dried over Na₂SO₄ and the solvent was evaporated. The crude product obtained was purified by chromatography on silica gel using CHCl₃-MeOH mixtures of increasing polarity as eluent, to afford 8 mg of the title compound (yield: 32%).

LC-MS (method 1): t_{R}=4.18 min; m/z=293.0 [M+H]⁺.

### EXAMPLE 17

### (1S)-1-(4-fluorophenyl)-5-[(1-phenylethyl)amino]pyrazolo[3,4-f]isoquinoline

To a solution of 5-bromo-1-(4-fluorophenyl)pyrazolo[3,4-f]isoquinoline (93 mg, 0.3 mmol, obtained in example 1) in toluene (2 mL) under argon, palladium acetate (II) (5 mg, 0.02 mmol), (±) 2,2′-bis(diphenylphosphino)-1,1′-binaphthyl (14 mg, 0.02 mmol) and sodium tert-butoxide (36 mg, 0.4 mmol) were added. Then, (S)-1-(phenyl)ethylamine (36 mg, 0.3 mmol) was added and the mixture was heated at 100° C. overnight. It was allowed to cool and CH₂Cl₂ and water were added. The phases were separated. The aqueous phase was reextracted 2 times with CH₂Cl₂. The combined organic phases were dried over Na₂SO₄ and the solvent was evaporated. The crude product obtained was purified by chromatography on silica gel using hexane-EtOAc mixtures of increasing polarity as eluent, to afford 65 mg of the title compound as an orange solid (yield: 63%).

LC-MS (method 1): t_{R}=7.80 min; m/z=383.0 [M+H]⁺.

### EXAMPLES 18-22

Following a similar procedure to that described in example 17, but starting from the appropriate compounds in each case, the compounds in the following table were obtained:

| | | | LC-MS | | |
|---|---|---|---|---|---|
| | | | | t_{R} | m/z |
| Example | Compound name | Starting products | Method | (min) | [M + H]⁺ |
| 18 | 1-(4-Fluorophenyl)-5-(phenylamino)- | Example 1 and | 1 | 7.88 | 355.0 |
| | pyrazolo[3,4-f]isoquinoline | aniline | | | |
| | | Base: NaO^{t}Bu | | | |
| 19 | 1-(4-Fluorophenyl)-5-(morpholin-4- | Example 1 and | 1 | 5.93 | 349.1 |
| | yl)pyrazolo[3,4-f]isoquinoline | morpholine | | | |
| | | Base: Cs₂CO₃ | | | |
| 20 | 5-(4-Acetylpiperazin-1-yl)-1-(4- | Example 1 and 1- | 1 | 5.43 | 390.1 |
| | fluorophenyl)pyrazolo[3,4- | acetylpiperazine | | | |
| | f]isoquinoline | Base: Cs₂CO₃ | | | |
| 21 | 1-(4-Fluorophenyl)-5-(4- | Example 1 and 1- | 1 | 3.97 | 362.1 |
| | methylpiperazin-1-yl)pyrazolo[3,4- | methylpiperazine | | | |
| | f]isoquinoline | Base: Cs₂CO₃ | | | |
| 22 | [1-(4-Fluorophenyl)pyrazolo[3,4- | Example 1 and | 1 | 5.93 | 361.0 |
| | f]isoquinolin-5-yl]piperidin-4-one | piperidin-4-one | | | |
| | | Base: Cs₂CO₃ | | | |

### EXAMPLE 23

### 1-(4-Fluorophenyl)pyrazolo[3,4-f]isoquinolin-5-carboxamide

To a suspension of 1-(4-fluorophenyl)pyrazolo[3,4-f]isoquinolin-5-carbonitrile (65 mg, 0.2 mmol, obtained in example 15) in tert-butanol (4 mL), KOH (126 mg, 2.2 mmol) was added and the mixture was heated at reflux for 3 h. It was allowed to cool and CHCl₃ and water were added. The phases were separated. The aqueous phase was reextracted 2 times with CHCl₃. The combined organic phases were washed with brine, dried over Na₂SO₄ and the solvent was evaporated. The crude product obtained was purified by chromatography on silica gel using EtOAc-MeOH mixtures of increasing polarity as eluent, to afford 38 mg of the title compound (yield: 56%).

LC-MS (method 1): t_{R}=4.45 min; m/z=307.0 [M+H]⁺.

### EXAMPLE 24

### 1-(4-Fluorophenyl)-5-(3-pyridyl)pyrazolo[3,4-f]isoquinolin-3-carboxamide

### a) 1-(4-Fluorophenyl)-5-(3-pyridyl)pyrazolo[3,4-f]isoquinolin-3-carbonitrile

Following a similar procedure to that described in example 15 but starting from 3-bromo-1-(4-fluorophenyl)-5-(3-pyridyl)pyrazolo[3,4-f]isoquinoline (obtained in example 11), the desired compound was obtained.

LC-MS (method 1): t_{R}=7.49 min; m/z=366.0 [M+H]⁺.

### b) Title Compound

Following a similar procedure to that described in example 23 but starting from 1-(4-fluorophenyl)-5-(3-pyridyl)pyrazolo[3,4-f]isoquinolin-3-carbonitrile (obtained in section a), the title compound was obtained.

LC-MS (method 1): t_{R}=4.88 min; m/z=384.0 [M+H]⁺.

### EXAMPLE 25

### 1-(4-Fluorophenyl)-3-[(4-methylsulfinylbenzyl)amino]-5-(3-pyridyl)pyrazolo[3,4-f]isoquinoline

### a) 1-(4-Fluorophenyl)-3-[(4-methylsulfanylbenzyl)amino]-5-(3-pyridyl)pyrazolo[3,4-f]isoquinoline

To a suspension of 3-amino-1-(4-fluorophenyl)-5-(3-pyridyl)pyrazolo[3,4-f]isoquinoline (100 mg, 0.3 mmol, obtained in example 14) in 1,2-dichloroethane (5 mL) under argon, a solution of 4-methylsulfanylbenzaldehyde (67 mg, 0.4 mmol) in 1,2-dichloroethane (0.5 mL) and sodium triacetoxyborohydride (239 mg, 1.2 mmol) were successively added. The mixture was stirred at room temperature for 3 days. The solvent was evaporated and CH₂Cl₂ was added. It was washed 2 times with saturated NaHCO₃. The aqueous phase was reextracted 2 times with CH₂Cl₂. The combined organic phases were washed with brine, dried over Na₂SO₄ and the solvent was evaporated. The crude product obtained was purified by chromatography on silica gel using hexane-EtOAc mixtures of increasing polarity as eluent, to afford 73 mg of the desired compound (yield: 53%).

LC-MS (method 1): t_{R}=8.68 min; m/z=492.0 [M+H]⁺.

### b) Title Compound

To a solution of 1-(4-fluorophenyl)-3-[(4-methylsulfanylbenzyl)amino]-5-(3-pyridyl)pyrazolo[3,4-f]isoquinoline (0.06 g, 0.1 mmol, obtained in section a) in CH₂Cl₂ (5 mL), m-chloroperbenzoic acid (28 mg 77%, 0.1 mmol) was added and the mixture was stirred at room temperature for 1 h 30 min. It was diluted with CH₂Cl₂ and the organic phase was washed 2 times with saturated NaHCO₃. The phases were separated. The aqueous phase was reextracted 2 times with CH₂Cl₂. The combined organic phases were dried over Na₂SO₄ and the solvent was evaporated. The crude product obtained was purified by chromatography on silica gel using EtOAc-MeOH mixtures of increasing polarity as eluent, to afford 41 mg of the title compound (yield: 68%).

LC-MS (method 1): t_{R}=5.60 min; m/z=508.0 [M+H]⁺.

### EXAMPLE 26

### 1-(4-Fluorophenyl)-3-(4-methylsufinylphenyl)-5-(3-pyridyl)pyrazolo[3,4-f]isoquinoline

### a) 1-(4-Fluorophenyl)-3-(4-methylsulfanylphenyl)-5-(3-pyridyl)pyrazolo[3,4-f]isoquinoline

Following a similar procedure to that described in example 2, but starting from 3-bromo-1-(4-fluorophenyl)-5-(3-pyridyl)pyrazolo[3,4-f]isoquinoline (obtained in example 11) and (4-methylsulfanylphenyl)boronic acid, the desired compound was obtained.

LC-MS (method 1): t_{R}=9.80 min; m/z=463.0 [M+H]⁺.

### b) Title Compound

Following a similar procedure to that described in example 25 section b, but starting from 1-(4-fluorophenyl)-3-(4-methylsulfanylphenyl)-5-(3-pyridyl)pyrazolo[3,4-f]isoquinoline (obtained in section a), the title compound was obtained.

LC-MS (method 1): t_{R}=6.09 min; m/z=479.0 [M+H]⁺.

### EXAMPLE 27

### 1-(4-Fluorophenyl)-5-(piperazin-1-yl)pyrazolo[3,4-f]isoquinoline

### a) 5-[4-(tert-Butoxycarbonyl)piperazin-1-yl]-1-(4-fluorophenyl)pyrazolo[3,4-f]isoquinoline

Following a similar procedure to that described in example 17, but starting from 5- bromo-1-(4-fluorophenyl)pyrazolo[3,4-f]isoquinoline (obtained in example 1) and 1-(tert-butoxycarbonyl)piperazine and using Cs₂CO₃ as base, the desired compound was obtained.

LC-MS (method 1): t_{R}=8.48 min; m/z=448.1 [M+H]⁺.

### b) Title Compound

To a solution of 5-[4-(tert-butoxycarbonyl)piperazin-1-yl]-1-(4-fluorophenyl)pyrazolo[3,4-f]isoquinoline (90 mg, 0.2 mmol, obtained in section a) in CH₂Cl₂ (4 mL) at 0° C., trifluoroacetic acid (0.33 mL) was added and the mixture was stirred at room temperature for 2 h. It was concentrated to dryness. CHCl₃ was added and it was washed with 0.5 N NaOH. The phases were separated. The aqueous phase was reextracted 2 times with CHCl₃. The combined organic phases were washed with brine, dried over Na₂SO₄ and the solvent was evaporated, to afford 70 mg of the title compound (yield: quantitative).

LC-MS (method 1): t_{R}=3.89 min; m/z=348.0 [M+H]⁺.

### EXAMPLE 28

### 1-(4-Fluorophenyl)-3-[(4-piperidylmethyl)amino]-5-(3-pyridyl)pyrazolo[3,4-f]isoquinoline

### a) 1-(4-Fluorophenyl)-3-[[N-(tert-butoxycarbonyl)piperidin-4-ylmethyl]amino]-5-(3-pyridyl)pyrazolo[3,4-f]isoquinoline

Following a similar procedure to that described in example 25 section a, but using 1-(tert-butoxycarbonyl)piperidin-4-carbaldehyde (J. Med. Chem. 1999, 5254-5265) instead of 4-methylsulfanylbenzaldehyde, the desired compound was obtained.

LC-MS (method 1): t_{R}=8.76 min; m/z=553.0 [M+H]⁺.

### b) Title Compound

Following a similar procedure to that described in example 27 section b, but starting from 1-(4-fluorophenyl)-3-[[N-(tert-butoxycarbonyl)piperidin-4-ylmethyl]amino]-5-(3-pyridyl)pyrazolo[3,4-f]isoquinoline (obtained in section a), the title compound was obtained.

LC-MS (method 1): t_{R}=3.97 min; m/z=453.0 [M+H]⁺.

### EXAMPLE 29

### 1-(4-Fluorophenyl)-5-phenyl-3-[(4-piperidyl)aminomethyl]pyrazolo[3,4-f]isoquinoline

### a) 5-Bromo-1-(4-fluorophenyl)pyrazolo[3,4-f]isoquinolin-3-carbaldehyde

Following a similar procedure to that described in example 8 section a, but starting from 1-(4-fluorophenyl)-4,5-dihydropyrazolo[3,4-f]isoquinolin-3-carbaldehyde (obtained in reference example 11), the title compound was obtained.

¹H NMR (300 MHz, CDCl₃) δ (TMS): 7.29 (d, J=6.0 Hz, 1 H), 7.41 (t, J=8.4 Hz, 2 H), 7.60-7.67 (complex signal, 2 H), 8.59 (d, J=5.7 Hz, 1 H), 8.81 (s, 1 H), 9.80 (s, 1 H), 10.34 (s, 1 H).

### b) 1-(4-Fluorophenyl)-5-phenylpyrazolo[3,4-f]isoquinolin-3-carbaldehyde

Following a similar procedure to that described in example 2, but starting from 5-bromo-1-(4-fluorophenyl)pyrazolo[3,4-f]isoquinolin-3-carbaldehyde (obtained in section a) and phenylboronic acid, the desired compound was obtained.

¹H NMR (300 MHz, CDCl₃) δ (TMS): 7.32-7.50 (complex signal, 3 H), 7.55 (m, 2 H), 7.62-7.70 (complex signal, 2 H), 8.42 (s, 1 H), 8.53 (d, J=5.8 Hz, 1 H), 9.34 (s, 1H), 10.37 (s, 1 H).

### c) 3-[[1-(tert-Butoxycarbonyl)piperidin-4-yl]aminomethyl]-1-(4-fluorophenyl)-5-phenylpyrazolo[3,4-f]isoquinoline

Following a similar procedure to that described in example 25 section a, but starting from 1-(4-fluorophenyl)-5-phenylpyrazolo[3,4-f]isoquinolin-3-carbaldehyde (obtained in section b) and 4-amino-1-(tert-butoxycarbonyl)piperidine (J. Med. Chem. 2001, 4404-4415), the desired compound was obtained.

¹H NMR (300 MHz, CDCl₃) δ (TMS): 1.32-1.40 (m, 2 H), 1.45 (s, 9 H), 1.93 (m, 2 H), 2.75-2.90 (complex signal, 3 H), 4.03 (m, 2 H), 4.30 (s, 2 H), 7.27-7.71 (complex signal, 10 H), 7.83 (s, 1 H), 8.46 (d, J=6.0 Hz, 1 H), 9.28 (s, 1 H).

### d) Title Compound

Following a similar procedure to that described in example 27 section b, but starting from 3-[[1-(tert-butoxycarbonyl)piperidin-4-yl]aminomethyl]-1-(4-fluorophenyl)-5-phenylpyrazolo[3,4-f]isoquinoline (obtained in section c), the title compound was obtained.

LC-MS (method 3): t_{R}=4.43 min; m/z=452.1 [M+H]⁺.

### EXAMPLE 30

### 1-[4-[1-(4-Fluorophenyl)pyrazolo[3,4-f]isoquinolin-5-yl]piperazin-1-yl]-2-hydroxyethanone

To a solution of 2-hydroxyacetic acid (23 mg, 0.3 mmol) in DMF (2 mL), 1,3-dicyclohexylcarbodiimide (69 mg, 0.3 mmol) and 1-hydroxybenzotriazole (45 mg, 0.3 mmol) were added. The mixture was stirred at room temperature for 45 min. A solution of 1-(4-fluorophenyl)-5-(piperazin-1-yl)pyrazolo[3,4-f]isoquinoline (105 mg, 0.3 mmol, obtained in example 27) in DMF (0.2 mL) was added. The mixture was stirred at room temperature overnight. The precipitated solid was filtered off and filtrate was concentrated. The resulting residue was taken up in CHCl₃ and was washed with saturated NaHCO₃. The aqueous phase was reextracted 2 times with CHCl₃. The combined organic phases were dried over Na₂SO₄ and the solvent was evaporated. The crude product obtained was purified by chromatography on silica gel using EtOAc-MeOH—NH₃ mixtures of increasing polarity as eluent, to afford 51 mg of the title compound (yield: 42%).

LC-MS (method 1): t_{R}=5.08 min; m/z=406.1 [M+H]⁺.

### EXAMPLE 31

### 4-[1-(4-Fluorophenyl)pyrazolo[3,4-f]isoquinolin-5-yl]phenol

A suspension of 1-(4-fluorophenyl)-5-[4-(tetrahydropyran-2-yloxy)phenyl]pyrazolo[3,4-f]isoquinoline (0.20 g, 0.5 mmol, obtained in example 2) in a mixture of HOAc: THF: H₂O (10 mL, 4:2:1) was heated at 55° C. overnight. It was allowed to cool and the solvent was evaporated. CHCl₃, MeOH and saturated NaHCO₃ were added. The phases were separated. The aqueous phase was extracted 3 times with CHCl₃. The combined organic phases were dried over Na₂SO₄ and the solvent was evaporated. The obtained solid was suspended in EtOAc, filtered, washed with diethyl ether and dried, to afford 124 mg of the title compound (yield: 77%).

LC-MS (method 1): t_{R}=6.56 min; m/z=356.0 [M+H]⁺.

### EXAMPLE 32

### [4-[1-(4-Fluorophenyl)pyrazolo[3,4-f]isoquinolin-5-yl]phenyl]methanol

### a) 4-[1-(4-Fluorophenyl)pyrazolo[3,4-f]isoquinolin-5-yl]benzaldehyde

Following a similar procedure to that described in example 2, but starting from 5-bromo-1-(4-fluorophenyl)pyrazolo[3,4-f]isoquinoline (obtained in example 1) and (4-formylphenyl)boronic acid, the desired compound was obtained.

¹H NMR (300 MHz, CDCl₃) δ (TMS): 7.36 (t, J=8.5 Hz, 2 H), 7.44 (d, J=5.7 Hz, 1 H), 7.63 (complex signal, 2 H), 7.73 (d, J=8.1 Hz, 2 H), 7.83 (s, 1 H), 8.07 (d, J=8.1 Hz, 2 H), 8.34 (s, 1 H), 8.51 (d, J=6.0 Hz, 1 H), 9.25 (s, 1H), 10.16 (s, 1 H).

### b) Title Compound

To a solution of 4-[1-(4-fluorophenyl)pyrazolo[3,4-f]isoquinolin-5-yl]benzaldehyde (86 mg, 0.2 mmol, obtained in section a) in EtOH (2 mL) and THF (3 mL), sodium borohydride (17 mg, 0.5 mmol) was added portionwise. The mixture was stirred at room temperature for 6 h. Water (3 mL) was added to the reaction and the solvent was evaporated. Water and EtOAc were added. The phases were separated and the aqueous phase was extracted with EtOAc. The combined organic phases were dried over Na₂SO₄ and the solvent was evaporated, to afford 78 mg of the title compound (yield: 90%).

LC-MS (method 1): t_{R}=6.42 min; m/z=370.0 [M+H]⁺

### EXAMPLE 33

### 3-(1,1-Dioxothiomorpholin-4-yl)-1-(4-fluorophenyl)-5-(3-pyridyl)pyrazolo[3,4-f]isoquinoline

To a mixture of 3-amino-1-(4-fluorophenyl)-5-(3-pyridyl)pyrazolo[3,4-f]isoquinoline (100 mg, 0.3 mmol, obtained in example 14) and vinyl sulfone (99 mg, 0.9 mmol), 85% H₃PO₄ (1 mL) was added and the mixture was heated at 140° C. for a week. It was allowed to cool and water and 30%NH₃ were added until pH =8-9. It was extracted with CHCl₃. The combined organic phases were dried over Na₂SO₄ and the solvent was evaporated. The crude product obtained was purified by chromatography on silica gel using EtOAc-MeOH—NH₃ mixtures of increasing polarity as eluent, to afford 6 mg of the title compound (yield: 4%).

LC-MS (method 1): t_{R}=5.92 min; m/z=474.0 [M+H]⁺.

### EXAMPLE 34

### 1-(4-Fluorophenyl)-3-(4-piperidylamino)-5-(3-pyridyl)pyrazolo[3,4-f]isoquinoline

### a) 1-(4-Fluorophenyl)-3-[1-(benzyloxycarbonyl)piperidin-4-ylamino]-5-(3-pyridyl)pyrazolo[3,4-f]isoquinoline

Following a similar procedure to that described in example 25 section a, but starting from 3-amino-1-(4-fluorophenyl)-5-(3-pyridyl)pyrazolo[3,4-f]isoquinoline (obtained in example 14) and 1-benzyloxycarbonylpiperidin-4-one, the desired compound was obtained.

¹H NMR (300 MHz, CDCl₃) δ (TMS): 7.36 (t, J=8.5 Hz, 2 H), 7.44 (d, J=5.7 Hz, 1 H), 7.63 (complex signal, 2 H), 7.73 (d, J=8.1 Hz, 2 H), 7.83 (s, 1 H), 8.07 (d, J=8.1 Hz, 2 H), 8.34 (s,1 H), 8.51 (d, J=6.0 Hz, 1 H), 9.25 (s, 1H), 10.16 (s, 1 H).

### b) Title Compound

To a solution of 1-(4-fluorophenyl)-3-[1-(benzyloxycarbonyl)piperidin-4-ylamino]-5-(3-pyridyl)pyrazolo[3,4-f]isoquinoline (50 mg, 0.1 mmol obtained in section a) in MeOH (1 mL) under argon, 10% Pd/C (6 mg) and a solution of ammonium formate (22 mg, 0.3 mmol) in water (0.15 mL) were added. The mixture was heated at reflux for 2 h. It was allowed to cool and diluted with MeOH. It was filtered through celite and washed with MeOH. The filtrate was evaporated and the residue was dissolved in a mixture of CHCl₃-MeOH and saturated NaHCO₃. The aqueous phase was reextracted 3 times with CHCl₃. The combined organic phases were dried over Na₂SO₄ and the solvent was evaporated. The crude product obtained was purified by chromatography on silica gel using CHCl₃-MeOH—NH₃ mixtures of increasing polarity as eluent, to afford 24 mg of the title compound (yield: 64%).

LC-MS (method 1): t_{R}=3.82 min; m/z=439.0 [M+H]⁺.

### EXAMPLE 35

### 5-[4-Acetylpiperazin-1-yl]-1-(3-trifluoromethylphenyl)pyrazolo[3,4-f]isoquinoline

Following a similar procedure to that described in example 17, but starting from 5-bromo-1-(3-trifluoromethylphenyl)pyrazolo[3,4-f]isoquinoline (obtained in example 10 section a), 1-acetylpiperazine and Cs₂CO₃ instead of ^{t}BuONa, the title compound was obtained.

LC-MS (method 1): t_{R}=6.44 min; m/z=440.2 [M+H]⁺.

### EXAMPLE 36

### 5-[4-Methylpiperazin-1-yl]-1-(3-trifluoromethylphenyl)pyrazolo[3,4-f]isoquinoline

To a solution of 5-bromo-1-(3-trifluoromethylphenyl)pyrazolo[3,4-f]isoquinoline (48 mg, 0.12 mmol, obtained in example 10 section a) in toluene (2 mL) under argon, tris(dibenzylideneacetone)palladium (0) (13 mg, 0.01 mmol), (±) 2,2′-bis(diphenylphosphino)-1,1′-binaphthyl (4.5 mg, 0.01 mmol) and Cs₂CO₃ (60 mg, 0.18 mmol) were added. Then, a solution of 1-methylpiperazine (15 mg, 0.15 mmol) in toluene (0.5 mL) was added and the mixture was heated at 110° C. overnight. It was allowed to cool and CH₂Cl₂ and water were added. The phases were separated. The aqueous phase was reextracted 2 times with CH₂Cl₂. The combined organic phases were washed with brine, dried over Na₂SO₄ and the solvent was evaporated. The crude product obtained was purified by chromatography on silica gel using EtOAc-NH₃ mixtures of increasing polarity as eluent, to afford 4.7 mg of the title compound (yield: 9%).

LC-MS (method 1): t_{R}=4.68 min; m/z=412.2 [M+H]⁺.

1. A compound of general formula I 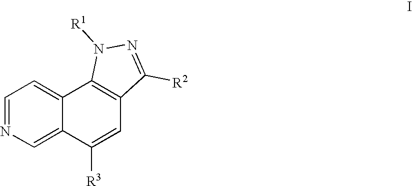
wherein:
R¹ represents phenyl optionally substituted with one or more substituents selected from R¹, halogen, —CN, —OH and —OR^{a};
R² represents H, halogen, —OR^{b′}, —NO₂, —CN, —COR^{b′}, —CO₂R^{b′}, —CONR^{b′}R^{c′}, NR^{b′}R^{d}, —NR^{c′}COR^{b′}, —NR^{c′}CONR^{b′}R^{c′}, —NR^{c′}CO₂ R^{b}, —NR^{c′}SO₂R^{b}, Cy¹, —(C₁₋₄alkyl)-Cy¹ or C₁₋₄alkyl optionally substituted with one or more substituents selected from halogen, —OR^{e′}, —NO₂, —CN, —COR^{e′}, —CO₂R^{e′}, —CONR^{c′}R^{e′}, —NR^{d}R^{e′}, —NR^{c′}COR^{e′}, —NR^{c′}CONR^{c′}R^{e′}, —NR^{c′}CO₂R^{e} and —NR^{c′}SO₂R^{e};
R³ represents halogen, —OR^{f′}, —NO₂, —CN, —COR^{f′}, —CO₂R^{f′}, —CONR^{c′}R^{f′}, —NR^{d}R^{f′}, —NR^{c′}COR^{f′}, —NR^{c′}CONR^{c′}R^{f′}, —NR^{c′}CO₂R^{f}, —NR^{c′}SO₂R^{f}, Cy², —(C₁₋₄alkyl)-Cy¹ or —(C₁₋₄alkyl) —NR^{c′}R^{f′};
Cy¹ represents Cy optionally substituted with one or more substituents selected from R^{c} and R^{g};
Cy² represents Cy optionally substituted with one or more substituents selected from R^{b} and R^{h};
each R^{a} independently represents C₁₋₄alkyl or haloC₁₋₄alkyl;
each R^{b} independently represents Cy¹, —(C₁₋₄alkyl)-Cy¹ or C₁₋₄alkyl optionally substituted with one or more substituents R^{g};
each R^{b′} independently represents H or R^{b};
each R^{c} independently represents C₁₋₄alkyl, haloC₁₋₄alkyl or hydroxyC₁₋₄alkyl;
each R^{c′} independently represents H or R^{c};
each R^{d} independently represents R^{c′} or —COR^{c};
each R^{e} independently represents R^{c} or Cy¹;
each R^{e′} independently represents H or R^{e};
each R^{f} independently represents R^{e} or —(C₁₋₄alkyl)-Cy¹;
each R^{f′} independently represents H or R^{f};
each R^{g} independently represents halogen, —OR^{c′}, —NO₂, —CN, —COR^{c′}, —CO₂R^{c′}, —CONR^{c′}R^{c′}, —NR^{c′}R^{c′}, —NR^{c′}COR^{c′}, —NR^{c′}CONR^{c′}R^{c′}, —NR^{c′}CO₂R^{c}, —NR^{c′}SO₂R^{c}, —SR^{c′}, —SOR^{c}, —SO₂R^{c} or —SO₂NR^{c′}R^{c′};
each R^{h} independently represents halogen, —OR^{b′}, —NO₂, —CN, —COR^{b′}, —CO₂R^{b′}, —CONR^{b′}R^{c′}, —NR^{b′}R^{d}, —NR^{c′}COR^{b′}, —NR^{c′}CONR^{b′}R^{c′}, —NR^{c′}, CO₂R^{b}, —NR^{c′}SO₂R^{b}; —SR^{b′}, —SOR^{b}, —SO₂R^{b} or —SO₂NR^{b′}R^{c′}; and
Cy in the above definitions represents a saturated, partially unsaturated or aromatic 3- to 7-membered monocyclic or 8- to 12-membered bicyclic carbocyclic ring, which optionally contains from 1 to 4 heteroatoms selected from N, S and O, wherein one or more C, N or S can be optionally oxidized forming CO, N⁺O⁻, SO or SO₂respectively, and wherein said ring or rings can be linked to the rest of the molecule through a carbon or a nitrogen atom; or a salt thereof.

2. A compound according to claim 1 wherein R¹ represents phenyl substituted with one or more substituents selected from halogen and haloC₁₋₄alkyl.

3. A compound according to claim 2 wherein R¹ represents phenyl substituted with one or more halogen atoms.

4. A compound according to claim 3 wherein R¹ represents phenyl substituted with one or two fluorine atoms.

5. A compound according to claim 1 wherein R² represents H, halogen, —CONR^{b′}R^{c′}, —NR^{b′}R^{d}, Cy¹ or C₁₋₄alkyl optionally substituted with one or more substituents selected from —OR^{e′} and —NR^{d}R^{e′}.

6. A compound according to claim 5 wherein R² represents H, —CONR^{b′}R^{c′}, —NR^{b′}R^{d}, Cy¹ or C₁₋₄alkyl substituted with one or more substituents selected from —OR^{e′} and —NR^{e′}R^{d}.

7. A compound according to claim 6 wherein R² represents H.

8. A compound according to claim 1 wherein R³ represents halogen, —CN, —CONR^{c′}R^{f′}, —NR^{d}R^{f′}, Cy² or —(C₁₋₄alkyl) —NR^{c′}R^{f′}.

9. A compound according to claim 8 wherein R³ represents —NR^{f′}R^{d} or Cy².

10. A compound according to claim 9 wherein R³ represents Cy².

11. A compound according to claim 10 wherein R³ represents a saturated, partially unsaturated or aromatic 6-membered monocyclic carbocyclic ring, which optionally contains 1 or 2 heteroatoms selected from N, S and O, and wherein one or more C, N or S atoms can be optionally oxidized forming CO, N⁺O⁻, SO or SO₂ respectively, wherein R³ can be optionally substituted with one or more substituents selected from R^{b} and R^{h}.

12. A compound according to claim 11 wherein R³ represents:
(i) an aromatic 6-membered carbocyclic ring optionally containing 1 or 2 N atoms, or
(ii) a saturated 6-membered heterocyclic ring containing 1 or 2 heteroatoms selected from N, S and O and wherein one or more C or S atoms can be optionally oxidized forming CO, SO or SO₂ respectively,
wherein R³ can be optionally substituted with one or more substituents selected from R^{b} and R^{h}.

13. A compound according to claim 12 wherein R³ represents morpholinyl, piperazinyl, 4-oxo-piperidinyl, phenyl or pyridyl, wherein R³ can be optionally substituted with one or more substituents selected from R^{b} and R^{h}.

14. A compound according to claim 1 selected from:
5-Bromo-1-(4-fluorophenyl)pyrazolo[3,4-f]isoquinoline;
1-(4-Fluorophenyl)-5-[4-(tetrahydropyran-2-yloxy)phenyl]pyrazolo[3,4-f]isoquinoline;
1-(4-Fluorophenyl)-5-(4-pyridyl)pyrazolo[3,4-f]isoquinoline;
1-(4-Fluorophenyl)-5-phenyl-pyrazolo[3,4-f]isoquinoline;
5-(2-Chlorophenyl)-1-(4-fluorophenyl)pyrazolo[3,4-f]isoquinoline;
1-(4-Fluorophenyl)-5-(3-pyridyl)-pyrazolo[3,4-f]isoquinoline;
5-(4-Aminophenyl)-1-(4-fluorophenyl)pyrazolo[3,4-f]isoquinoline;
[1-(4-Fluorophenyl)-5-(3-pyridyl)pyrazolo[3,4-f]isoquinolin-3-yl]methanol;
[1-(4-Fluorophenyl)-5-phenylpyrazolo[3,4-f]isoquinolin-3-yl]methanol;
5-(3-Pyridyl)-1-(3-trifluoromethylphenyl)pyrazolo[3,4-f]isoquinoline;
3-Bromo-1-(4-fluorophenyl)-5-(3-pyridyl)pyrazolo[3,4-f]isoquinoline;
3-Aminomethyl-1-(4-fluorophenyl)-5-phenylpyrazolo[3,4-f]isoquinoline;
4-[1-(4-Fluorophenyl)pyrazolo[3,4-f]isoquinolin-5-yl]benzoic acid;
3-Amino-1-(4-fluorophenyl)-5-(3-pyridyl)pyrazolo[3,4-f]isoquinoline;
1-(4-Fluorophenyl)pyrazolo[3,4-f]isoquinolin-5-carbonitrile;
5-Aminomethyl-1-(4-fluorophenyl)pyrazolo[3,4-f]isoquinoline; (1S)-1-(4-fluorophenyl)-5-[(1-phenylethyl)amino]pyrazolo[3,4-f]isoquinoline;
1-(4-Fluorophenyl)-5-(phenylamino)-pyrazolo[3,4-f]isoquinoline;
1-(4-Fluorophenyl)-5-(morpholin-4-yl)pyrazolo[3,4-f]isoquinoline;
5-(4-Acetylpiperazin-1-yl)-1-(4-fluorophenyl)pyrazolo[3,4-f]isoquinoline;
1-(4-Fluorophenyl)-5-(4-methylpiperazin-1-yl)pyrazolo[3,4-f]isoquinoline;
[1-(4-Fluorophenyl)pyrazolo[3,4-f]isoquinolin-5-yl]piperidin-4-one;
1-(4-Fluorophenyl)pyrazolo[3,4-f]isoquinolin-5-carboxamide;
1-(4-Fluorophenyl)-5-(3-pyridyl)pyrazolo[3,4-f]isoquinolin-3-carboxamide;
1-(4-Fluorophenyl)-3-[(4-methylsulfinylbenzyl)amino]-5-(3-pyridyl)pyrazolo[3,4-f]isoquinoline;
1-(4-Fluorophenyl)-3-(4-methylsulfinylphenyl)-5-(3-pyridyl)pyrazolo[3,4-f]isoquinoline;
1-(4-Fluorophenyl)-5-(piperazin-1-yl)pyrazolo[3,4-f]isoquinoline;
1-(4-Fluorophenyl)-3-[(4-piperidylmethyl)amino]-5-(3-pyridyl)pyrazolo[3,4-f]isoquinoline;
1-(4-Fluorophenyl)-5-phenyl-3-[(4-piperidyl)aminomethyl]pyrazolo[3,4-f]isoquinoline;
1-[4-[1-(4-Fluorophenyl)pyrazolo[3,4-f]isoquinolin-5-yl]piperazin-1-yl]-2-hydroxyethanone;
4-[1-(4-Fluorophenyl)pyrazolo[3,4-f]isoquinolin-5-yl]phenol;
[4-[1-(4-Fluorophenyl)pyrazolo[3,4-f]isoquinolin-5-yl]phenyl]methanol;
3-(1,1-Dioxothiomorpholin-4-yl)-1-(4-fluorophenyl)-5-(3-pyridyl)pyrazolo[3,4-f]isoquinoline;
1-(4-Fluorophenyl)-3-(4-piperidylamino)-5-(3-pyridyl)pyrazolo[3,4-f]isoquinoline;
5-[4-Acetylpiperazin-1-yl]-1-(3-trifluoromethylphenyl)pyrazolo[3,4-f]isoquinoline; and
5-[4-Methylpiperazin-1-yl]-1-(3-trifluoromethylphenyl)pyrazolo[3,4-f]isoquinoline.

15. A process for the preparation of a compound of formula I according to claim 1, which comprises:
(a) when in a compound of formula I R³ represents halogen, reacting a compound of formula IV 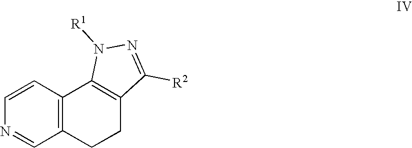
wherein R¹ and R² have the meaning described in claim 1, with a suitable halogenating agent; or
(b) when in a compound of formula I R³ represents aryl or heteroaryl optionally substituted with one or more substituents selected from R^{b} and R^{h}, reacting a compound of formula I wherein R³ represents halogen (Ia) 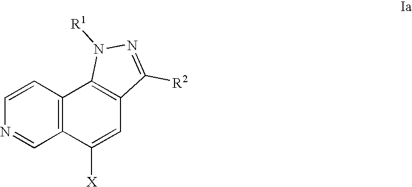
wherein R¹ and R² have the meaning described in claim 1 and X represents halogen, preferably chloro or bromo, with a boron derivative of formula Cy²-B(ORⁱ)₂ (II) or with a derivative of formula IIa, 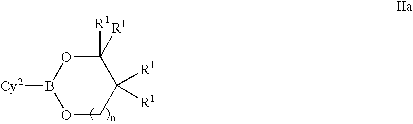
wherein n represents 0 or 1, Cy² represents aryl or heteroaryl optionally substituted with one or more substituents selected from R^{b} and R^{h}, and wherein each Rⁱ independently represents H or C₁₋₄alkyl; or
(c) when in a compound of formula I R³ represents —NR^{f}R^{c′}, reacting a compound of formula Ia with an amine of formula HNR^{f}R^{c′} (III); or
(d) when in a compound of formula I R³ represents Cy² linked to the central ring through a N atom, reacting a compound of formula Ia with the corresponding cyclic amine; or
(e) converting, in one or a plurality of steps, a compound of formula I into another compound of formula I.

16. A pharmaceutical composition which comprises a compound of formula I according to claim 1 or a pharmaceutically acceptable salt thereof and one or more pharmaceutically acceptable excipients.

17-18. (canceled)

19. A method of treating or preventing a disease mediated by p38 which comprises administering to a subject in need thereof an effective amount of a medicament comprising a compound of claim 1 or a pharmaceutically acceptable salt thereof.

20. A method according to claim 19 wherein the disease mediated by p38 is selected from immune diseases, autoimmune diseases, inflammatory diseases, cardiovascular diseases, infectious diseases, bone resorption diseases, neurodegenerative diseases, proliferative diseases and processes associated with the induction of cyclooxygenase-2.
